(19) United States
(12) Patent Application Publication
(10) Pub. No.: US 2021/0186978 A1
(43) Pub. Date: Jun. 24, 2021

(54) COMBINATION THERAPY

(71) Applicant: TARVEDA THERAPEUTICS, INC., Watertown, MA (US)

(72) Inventors: Kerry WHALEN, Waltham, MA (US); Samantha PERINO, Brighton, MA (US); Richard WOOSTER, Natick, MA (US); Sudhakar KADIYALA, Newton, MA (US); James M. QUINN, Lynnfield, MA (US)

(21) Appl. No.: 15/734,110

(22) PCT Filed: May 31, 2019
(86) PCT No.: PCT/US19/34894

§ 371 Date: Dec. 1, 2020

Related U.S. Application Data
(60) Provisional application No. 62/679,216, filed on Jun. 1, 2018.
(60) Provisional application No. 62/788,326, filed on Jan. 4, 2019.
(60) Provisional application No. 62/827,232, filed on Apr. 1, 2019.

(51) Int. Cl. A61K 31/537; A61K 47/64; A61K 31/167; A61K 38/31; A61K 31/4745; A61K 31/4545; A61K 31/513; A61K 31/704; A61K 31/519; A61K 31/4184; A61K 31/565; A61K 31/454; A61K 45/06; A61P 35/00; A61K 47/54

(52) U.S. Cl. CPC A61K 31/537; A61K 47/545; A61K 31/167; A61K 38/31; A61K 31/4745; A61K 31/4545; A61K 31/513; A61K 31/704; A61K 31/519; A61K 31/4184; A61K 31/565; A61K 31/454; A61K 45/06; A61P 35/00; A61K 47/645

(57) ABSTRACT

The invention generally relates to a combination therapy for treating cancer comprising administering at least two distinct therapeutic agents. Components of the combination therapy and methods of using the combination therapy are provided.

### REFERENCED TO RELATED APPLICATIONS

The present application claims priority to U.S. Provisional Patent Application No. 62/679,216, filed Jun. 1, 2018, entitled COMBINATION THERAPY, U.S. Provisional Patent Application No. 62/788,326, filed Jan. 4, 2019, entitled COMBINATION THERAPY, and U.S. Provisional Patent Application No. 62/827,232, filed Apr. 1, 2019, entitled COMBINATION THERAPY, the contents of each of which are herein incorporated by reference in their entirety.

### FIELD OF THE DISCLOSURE

The invention generally relates to a combination therapy for treating cancer.

### BACKGROUND

Cancer is a malignant growth characterized by unregulated proliferation of cells. Cancerous cells propagate from a single cell and can be multiplied to develop into tumor tissues. The cancerous cells can invade nearby tissues and spread through the bloodstream and lymphatic system to other parts of the body (metastasis). Chemotherapeutic drugs such as alkylating agents (e.g. cyclophosphamide), antimetabolites (e.g. fluorouracil), plant alkaloids (e.g. paclitaxel), topoisomerase inhibitors (e.g. topotecan), and cytotoxic antibiotics (e.g. daunorubicin) have been developed to treat cancer. Most of these drugs impair cell division or DNA synthesis and functions. However, most of the chemotherapeutic drugs cause undesirable systemic effects such as cardiac or renal toxicity, marrow aplasia, alopecia, nausea and vomiting. Antibody drug conjugates comprise an antibody and a cytotoxic payload have been designed. However, the size of antibodies limits solid tumor penetration compared to smaller targeting ligands (see Xiang et al., Theranostics, vol. 5(10):1083-1097 (2015)). Accordingly, there is a need in the art for improved drug targeting and delivery and for therapies that have good outcome to treat cancer.

### SUMMARY

The present disclosure relates to a method of treating a patient with a hyperproliferative disorder such as cancer, comprising administering to the patient: (A) a first component which comprises, as an active agent, Component I, or a pharmaceutically-acceptable salt thereof; and (B) a second component which comprises, as an active agent, Component II, or a pharmaceutically-acceptable salt thereof the amounts of said active agents being such that the combination thereof is therapeutically-effective in the treatment of said hyperproliferative disorder. Component I may comprise a tubulin inhibitor and/or a targeting moiety that targets a somatostatin receptor (SSTR). Component II is different from Component I. Component II may comprise any active agent that targets heat shock protein 90 (HSP90), a checkpoint inhibitor, a radioactive agent, an Histone Deacetylase (HDAC) inhibitor, octreotide, or derivatives/analogs thereof.

The present disclosure further relates to a composition comprising: (A) a first component which comprises, as an active agent, Component I, or a pharmaceutically-acceptable salt thereof and (B) a second component which comprises, as an active agent, Component I, or a pharmaceutically-acceptable salt thereof.

The present disclosure also relates to a kit comprising: (A) a first component which comprises, as an active agent, Component I, or a pharmaceutically-acceptable salt thereof; and (B) a second component which comprises, as an active agent, Component II, or a pharmaceutically-acceptable salt thereof.

In addition, the present disclosure relates to the use of Component I, or a pharmaceutically-acceptable salt thereof, and Component II, or a pharmaceutically-acceptable salt thereof, for the treatment of a hyperproliferative disorder.

A yet further aspect of the present disclosure is the use of Component I, or a pharmaceutically-acceptable salt thereof, and Component II, or a pharmaceutically-acceptable salt thereof, for the preparation of a medicament for the treatment of a hyperproliferative disorder.

### BRIEF DESCRIPTION OF THE DRAWINGS

FIG. 1 shows NCI-H69 xenograft efficacy of vehicle, Conjugate 1 (Conj. 1) at 0.7 mg/kg, Conjugate 1 (Conj. 1) at 2 mg/kg, Conjugate 2 (Conj. 2) at 25 mg/kg, Combo 1 (Conjugate 1 at 0.7 mg/kg+Conjugate 2 at 25 mg/kg), and Combo 2 (Conjugate 2 at 25 mg/kg+Conjugate 1 at 0.7 mg/kg). Error bars indicate standard error of the mean.

FIG. 2 shows NCI-H69 xenograft efficacy of vehicle, Conjugate 1 (Conj. 1) at 0.7 mg/kg, vorinostat at 50 mg/kg and a combination therapy using Conjugate 1 and vorinostat.

FIG. 3 shows SSTR2 expressions in tumor tissues and normal tissues (spleen) after vorinostat treatment.

FIG. 4 shows Conjugate 1 potency with Vorinostat pre-treatment and without Vorinostat pre-treatment in vitro in NCI-H69 cells.

FIG. 5 shows Conjugate 1 tumor uptake with Vorinostat pre-treatment and without Vorinostat pre-treatment in mice bearing NCI-H69 tumors.

FIG. 6 shows tumor volumes in the NCI-H727 xenograft model after treatment with Conjugate 1 and Vorinostat in combination.

FIG. 7A (without Vorinostat pre-treatment) and FIG. 7B (with Vorinostat pre-treatment) show tumor volumes in NCI-H69 models after various treatment regimen.

### DETAILED DESCRIPTION

The present disclosure relates to a combination therapy of at least two distinct therapeutic agents for treating a hyperproliferative disorder such as cancer. Each distinct therapeutic agent is referred to as a “component” of the combination therapy. The combination therapy of the invention is highly effective in treating various types of cancer and shows enhanced effect compared to the activity of each of the components administered alone. The terms “combination therapy” or “combined treatment” or “in combination” as used herein refers to any form of concurrent or parallel treatment with at least two distinct therapeutic agents. A hyperproliferative disorder embraces any disease or malady characterized by uncontrolled cell proliferation.

The components of the combination therapy may be administered simultaneously, sequentially, or at any order. The components may be administered at different dosages, with different dosing frequencies, or via different routes, whichever is suitable.

The term “administered simultaneously” as used herein is not specifically restricted and means that the components of the combination therapy are substantially administered at the same time, e.g. as a mixture or in immediate subsequent sequence.

The term “administered sequentially” as used herein is not specifically restricted and means that the components of the combination therapy are not administered at the same time but one after the other, or in groups, with a specific time interval between administrations. The time interval may be the same or different between the respective administrations of the components of the combination therapy and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours. In some embodiments, Component I is administered before Component II. In some embodiments, Component II is administered before Component I.

The molar ratio of the components is not particularly restricted. For example, when two components are combined in a composition, the molar ratio between the two components may be in the range of 1:500 to 500:1, or of 1:100 to 100:1, or of 1:50 to 50:1, or of 1:20 to 20:1, or of 1:5 to 5:1, or 1:1. Similar molar ratios apply when more than two components are combined in a composition. Each component may comprise, independently, a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### I. Components in the Combination Therapy

One aspect of the present disclosure provides a combination therapy of treating a subject with a hyperproliferative disorder such as cancer, comprising administering to the patient: (A) a first component which comprises, as an active agent, Component I (or Compound I), or a prodrug, derivative, or pharmaceutically-acceptable salt thereof; and (B) a second component which comprises, as an active agent, Component II (or Compound II), or a prodrug, derivative, or a pharmaceutically-acceptable salt thereof; the amounts of said active agents being such that the combination thereof is therapeutically-effective in the treatment of said hyperproliferative disorder.

In some embodiments, Component I comprises a tubulin inhibitor or prodrug thereof. Component I may also be a small molecule conjugate comprising a tubulin inhibitor or prodrug thereof attached to a targeting moiety. The targeting moiety may bind to a SSTR.

Component II is different from Component I.

In some embodiments, Component II comprises a checkpoint inhibitor. A checkpoint inhibitor, as used herein, refers to an active agent that blocks immunosuppressive signals in the tumor microenvironment. In some embodiments, the active agent may be an antagonistic agent specific to a coinhibitory checkpoint molecule (e.g., CTLA-4, PD1, PD-L1) that can antagonize or reduce the inhibitory signal to effector immune cells. In some embodiments, the active agent may be an inhibitor that can inhibits and reduces the activity of immune suppressive enzymes (e.g. ARG and IDO) and cytokines (e.g. IL-10), chemokines and other soluble factors (e.g., TGF-β and VEGF) in the tumor microenvironment.

In some embodiments, Component II is radiation therapy and/or a radioactive agent.

In some embodiments, Component II is an active agent that targets heat shock protein 90 (HSP90).

In some embodiments, Component II is an HDAC inhibitor.

In some embodiments, Component II is octreotide or its derivative/analog

The term “small molecule” as used herein refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The term “targeting moiety” as used herein refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule such as a protein.

In some instances, a conjugate may have a molecular weight of less than about 50,000 Da, less than about 40,000 Da, less than about 30,000 Da, less than about 20,000 Da, less than about 15,000 Da, less than about 10,000 Da, less than about 8,000 Da, less than about 5,000 Da, or less than about 3,000 Da. In some cases, the conjugate may have a molecular weight of between about 1,000 Da and about 50,000 Da, between about 1,000 Da and about 40,000 Da, in some embodiments between about 1,000 Da and about 30,000 Da, in some embodiments bout 1,000 Da and about 50,000 Da, between about 1,000 Da and about 20,000 Da, in some embodiments between about 1,000 Da and about 15,000 Da, in some embodiments between about 1,000 Da and about 10,000 Da, in some embodiments between about 1,000 Da and about 8,000 Da, in some embodiments between about 1,000 Da and about 5,000 Da, and in some embodiments between about 1,000 Da and about 3,000 Da. The molecular weight of the conjugate may be calculated as the sum of the atomic weight of each atom in the formula of the conjugate multiplied by the number of each atom. It may also be measured by mass spectrometry, NMR, chromatography, light scattering, viscosity, and/or any other methods known in the art. It is known in the art that the unit of molecular weight may be g/mol, Dalton (Da), or atomic mass unit (amu), wherein 1 g/mol=1 Da=1 amu.

Component I and Component II may be administered simultaneously, sequentially, or at any order. They may be administered at different dosages, with different dosing frequencies, or via different routes, whichever is suitable.

In some embodiments, the combination therapy further comprises administering to the patient a cancer symptom relief drug. The symptom relief drug may reduce diarrhea or the side effects of chemotherapy or radiation therapy. Non-limiting examples of symptom relief drugs include: octreotide or lanreotide; interferon, cypoheptadine or any other antihistamines; and/or a symptom relief drug for carcinoid symdrome, such as telotristat or telotristat etiprate (LX1032, Lexicon®). Telotristat etiprate is telotristat's crystalline hippurate salt as disclosed in WO2013059146 to Chen et al., the contents of which are incorporated herein by reference in their entirety. Telotristat, its salts and crystalline forms can be obtained by methods known in the art (see U.S. Pat. No. 7,709,493 to Devasagayaraj et al., the contents of which are incorporated herein by reference in their entirety). Any other compound disclosed in U.S. Pat. No. 7,709,493 may be incorporated in the combination therapy.

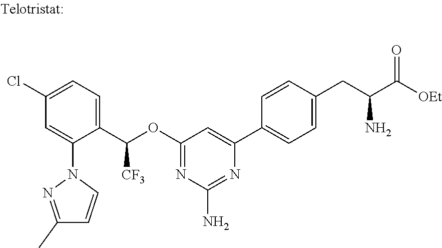 [SMILES: CCOC(=O)[C@@H](N)Cc1ccc(-c2cc(O[C@H](c3ccc(Cl)cc3-n3ccc(C)n3)C(F)(F)F)nc(N)n2)cc1]

### Component I

According to the present disclosure, Component I comprises a tubulin inhibitor that depolymerizes the microtubule assembly. In some embodiments, Component I is a conjugate comprising an active agent or prodrug thereof attached to a targeting moiety, wherein the active agent is a tubulin inhibitor.

In some embodiments, Component I is a conjugate comprising an active agent or prodrug thereof attached to a targeting moiety, wherein the active agent is a tubulin inhibitor and wherein the targeting moiety binds to a somatostatin receptor (SSTR), such as SSTR2. The active agent may be maytansine, maytansine derivatives such as DM1, dolastatin-10, or monomethyl auristatin E 04:MAE). The targeting moiety may be somatostatin, octreotide, seglitide, vapreotide, Tyr³-octreotate (TATE), cyclo(AA-Tyr-DTrp-Lys-Thr-Phe) where AA is α-N-Me lysine or N-Me glutamic acid, pasireotide, lanreotide, or analogs or derivatives thereof.

In some examples, Component I is a conjugate comprising DM1 or its prodrug thereof attached to a targeting moiety. DM1 binds to a site on β-tubulin and is a potent inhibitor of the microtubule assembly. Zippelius et al. have reported that a direct precursor of DM1, ansamitocin P3, is capable of inducing the full spectrum of maturation changes in dendritic cells (DCs), resulting in robust activation of antitumor immunity (Zipperlius et al., Science Translational Medicine, vol. 7(31):315 (2015)). They also demonstrated that ansamitocin P3 facilitates antigen uptake and migration of tumor resident DCs to the tumor-draining lymph nodes, thereby potentiating the tumor-specific T cell response in vivo.

In some examples, Component I is a conjugate comprising cyclo(AA-Tyr-DTrp-Lys-Thr-Phe) as a targeting moiety and DM1 as an active agent. Compound I may be any compound shown in Table 1 of PCT Application No. PCT/US15/38569 (WO2016/004048) filed Jun. 30, 2015, the contents of which are incorporated herein by reference.

In some examples, Component I is a conjugate comprising TATE or a TATE derivative as a targeting moiety and mertansine (DM1) as an active agent. Compound I may be any compound in Table 2 of PCT Application No. PCT/US15/38569 (WO2016/004048) filed Jun. 30, 2015, the contents of which are incorporated herein by reference.

In some examples, Component I may be any compound in Table 3 or Table 4 of PCT Application No. PCT/US15/38569 (WO2016/004048) filed Jun. 30, 2015, the contents of which are incorporated herein by reference.

In one example, Component I is Conjugate 1, a small molecule drug conjugate comprising TATE connected to DM1 via a cleavable disulfide linker, having a structure of

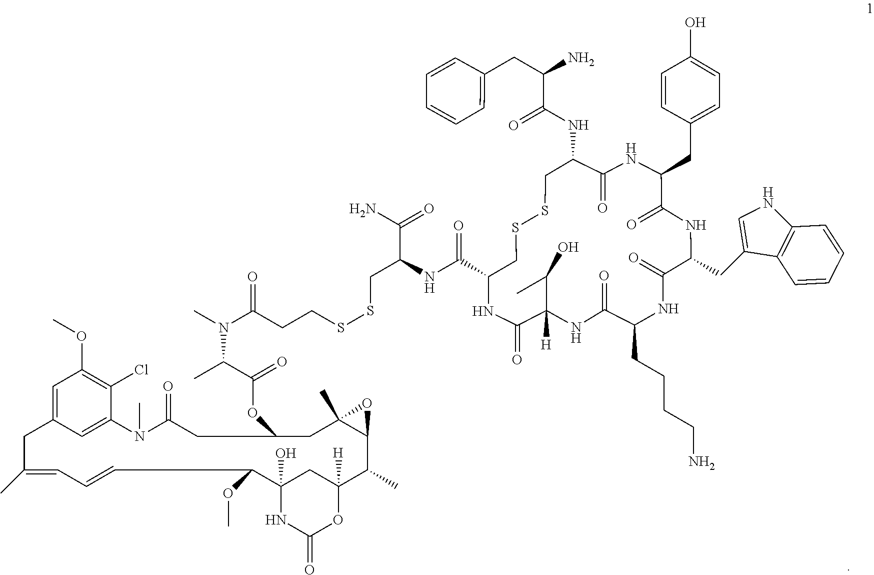 [SMILES: COc1cc2cc(c1Cl)N(C)C(=O)C[C@H](OC(=O)[C@H](C)N(C)C(=O)CCSSC[C@H](NC(=O)[C@@H]1CSSC[C@H](NC(=O)[C@H](N)Cc3ccccc3)C(=O)N[C@@H](Cc3ccc(O)cc3)C(=O)N[C@H](Cc3c[nH]c4ccccc34)C(=O)N[C@@H](CCCCN)C(=O)N[C@@H]([C@@H](C)O)C(=O)N1)C(N)=O)C[C@]1(C)O[C@H]1[C@H](C)[C@@H]1C[C@@](O)(NC(=O)O1)[C@H](OC)C/C=C/C=C(\C)C2]

In some embodiments, Conjugate 1 is administered in a pharmaceutical composition. The pharmaceutical composition may be administered via intravenous (IV) infusion. In some embodiments, the pharmaceutical composition may have a pH between about 4.0 to about 5.0. The pharmaceutical composition may comprise acetate buffer. The concentration of Conjugate 1 may be between about 2.0-about 5.0 mg/mL. In some embodiments, the pharmaceutical composition may further comprise mannitol. Mannitol may have a concentration of between about 25 to about 75 mg/mL. In some embodiments, the pharmaceutical composition may further comprise polyoxyl 15 hydroxystearate. Polyoxyl 15 hydroxystearate may have a concentration of between about 10 to about 50 mg/mL.

### Component II

Tumor cells can induce an immunosuppressive microenvironment to help them escape the immune surveillance. The immune suppression in the tumor microenvironment is either induced by intrinsic immune suppression mechanisms, or directly by tumors. Component II of the combination therapy comprises a checkpoint inhibitor that blocks such immunosuppressive signals in the tumor microenvironment.

In some embodiments, Component II may be an antagonistic agent specific to a coinhibitory checkpoint molecule that can antagonize or reduce the inhibitory signal to effector immune cells (e.g. cytotoxic T cells and natural killer cells).

In some embodiments, Component II may be an inhibitor that can inhibits and reduces the activity of immune suppressive enzymes (e.g. ARG and IDO) and cytokines (e.g. IL-10), chemokines and other soluble factors (e.g., TGF-β and VEGF) in the tumor microenvironment.

In some embodiments, Component II is selected from the group consisting of octreotide or its derivative/analog; cisplatin or its derivative/analog; etoposide or its derivative/analog; receptor tyrosine kinase inhibitors such as sunitinib or its derivative/analog; inhibitors of mammalian target of rapamycin (mTOR) such as everolimus or its derivative/analog; delta-like protein 3 (DLL3) inhibitors such as rovalpituzumab tesirine (ROVA-T) or its derivative/analog; poly ADP ribose polymerase (PARP) inhibitors such as talazoparib (BMN-673), olaparib (AZD-2281), niraparib (MK-4827), iniparib (BSI 201), veliparib (ABT-888), rucaparib (AG014699, PF-01367338), or CEP 9722; insulin-like growth factor receptor (IGFR) inhibitors that are small molecules or antibodies; protein kinase inhibitors such as lucaitanib; nintedanib (BIBF1120) or its derivative/analog; inhibitors of vascular endothelial growth factor (VEGF) receptors such as VEGFR1/2/3 inhibitors; inhibitors of fibroblast growth factor receptor (FGFR) such as FGFR1 inhibitors; platelet-derived growth factor (PDGF) A and B inhibitors; hepatocyte growth factor (HGF) inhibitors such as anti-HGF monoclonal antibodies (e.g., rilotumumab (i.e., AMG 102)); hedgehog pathway inhibitors such as vismodegib, sonidegib or itraconzaole; aurora kinase inhibitors such as ZM447439, hesperadin or VX-680; and CD56 targeting antibody drug conjugate such as lorvotuzumab mertansine comprising a humanized anti-CD56 antibody linked via a disulfide linker to maytansinoid (DM1).

In one embodiment, Component I is Conjugate 1 and Component II is octreotide or its derivative/analog. Component II may be administered to a subject before Conjugate 1 is administered.

### The Tumor Microenvironment

In adaptive immune responses for eliminating tumor cells, cytotoxic T cell activation needs both a primary signal from a specific antigen (i.e. first signal) and one or more co-stimulatory signals (i.e. secondary signal). Antigen presenting cells (APCs, e.g., dendritic cells (DCs)) process tumor associated antigens (TAAs) and present antigenic peptides derived from TAAs (i.e. epitopes) on the cell surface as peptide/MHC molecule (class I/II) (p/MHC) complexes and T cells engage APCs loaded with TAAs via their T cell receptors (TCRs) which recognize the p/MHC complexes. This ligation is the primary signal to activate cancer specific cytotoxic T cells. Additionally, a secondary co-stimulating signal is provided by co-stimulatory receptors on the T cells and their ligands (or coreceptors) on the APCs. The interaction between co-stimulatory receptors and their ligands can regulate T cell activation and enhance its activity. CD28, 4-1BB (CD137), and OX40 are well studied co-stimulatory receptors on T cells, which bind to B7-1/2 (CD80/CD86), 4-1BB (CD137L) and OX-40L, respectively on APCs. In normal circumstance, to prevent excessive T-cell proliferation and balance the immunity, a co-inhibitory signal, e.g., CTLA-4, can be induced and expressed by activated T cells and competes with CD28 in binding to B7 ligands on APCs. This can mitigate a T cell response in a normal circumstance. However, in some cancers, tumor cells and regulatory T cells infiltrating the tumor microenvironment can constitutively express CTLA-4. This co-inhibitory signal suppresses the co-stimulatory signal, therefore, depleting an anti-cancer immune response. This immune suppressing mechanism by tumor cells is referred to as an immune checkpoint or checkpoint pathway.

In addition to CTLA-4 signal, activated T cells can also be induced to express another inhibitory receptor, PD-1 (programed death 1). In normal situation, as an immune response progresses, CD4+ and CD8+T lymphocytes upregulate the expression of these inhibitory checkpoint receptors (e.g., PD-1). Inflammatory conditions prompt IFN release, which will upregulate the expression of PD-1 ligands: PD-L1 (also known as B7-H1) and PD-L2 (also known as B7-DC) in peripheral tissues, to maintain immune tolerance to prevent autoimmunity. Many human cancer types have been demonstrated to express PD-L1 in the tumor microenvironment (e.g., Zou and Chen, inhibitory B7-family in the tumor microenvironment. 2008, Nat Rev Immunol, 8: 467-477). The PD-1/PD-L1 interaction is highly active with the tumor microenvironment, inhibiting T cell activation.

Other identified co-inhibitory signals in the tumor microenvironment include TIM-3, LAG-3, BTLA, CD160, CD200R, TIGIT, KLRG-1, KIR, CD244/2B4, VISTA and Ara2R.

In addition, the tumor microenvironment contains suppressive elements including regulatory T cells (Treg), myeloid-derived suppressor cells (MDSC) and tumor-associated macrophage (TAM); soluble factors such as interleukin 6 (IL-6), IL-10, vascular endothelial growth factor (VEGF), and transforming growth factor beta (TGF-β). An important mechanism by which IL-10, TGF-β, and VEGF counteract the development of an anti-cancer immune response is through inhibition of dendritic cell (DC) differentiation, maturation, trafficking, and antigen presentation (Gabrilovich D: Mechanisms and functional significance of tumour-induced dendritic-cell defects, Nat Rev Immunol, 2004, 4: 941-952).

Regulatory T cells (Treg): CD4+CD25+ Treg cells represent a unique population of lymphocytes that are thymus-derived. CD4+CD25+ Treg cells, which were marked by forkhead box transcription factor (Foxp3), play a critical role in maintaining self-tolerance, suppress autoimmunity and regulate immune responses in organ transplantation and tumor immunity. Tumor development often attracts CD4+CD25+ FoxP3+ Treg cells to the tumor area. Tumor infiltrating regulatory T cells secret inhibitory cytokines such as IL-10 and TGFβ to inhibit autoimmune and chronic inflammatory responses and to maintain immune tolerance in tumors (Unitt et al., Compromised lymphocytes infiltrate hepatocellular carcinoma: the role of T-regulatory cells. Hepatology. 2005; 41(4):722-730).

Myeloid derived suppressor cells (MDSCs): MDSCs are a group of heterogeneous cells, which could be seen as hallmark of malignancy-associated inflammation and a major mediator for the induction of T cell suppression in cancers. MDSCs are found in many malignant areas and divided phenotypically into granulocytic (G-MDSC) and monocytic (Mo-MDSC) subgroups. MDSCs can induce T regulatory cells, and produce T cell tolerance. Additionally, MDSCs secrete TFG-β and IL-10 and produce nitric oxide (NO) in the presence of IFN-γ or activated T cells.

Tumor associated macrophage (TAM): TAMs derived from peripheral blood monocytes are multi-functional cells which exhibit different functions to different signals from the tumor microenvironment. Among cell types associated with tumor microenvironment, TAMs are the most influential for tumor progression. In response to microenvironmental stimuli, such as tumor extracellular matrix, anoxic environment and cytokines secreted by tumor cells, macrophages undergo M1 (classical) or M2 (alternative) activation. In most malignant tumors, TAMs have the phenotype of M2 macrophages.

Another immune suppressive mechanism relates to tryptophan catabolism by the enzyme indoleamine-2,3-dioxygenase (IDO). Local immune suppression is an active process induced by the malignant cells within the tumor microenvironment and within the sentinel lymph nodes (SLN). (Gajewski et al., Immune suppression in the tumor microenvironment. J Immunother, 2006; 29(3):233-240; and Zou W., Immunosuppressive networks in the tumor environment and their therapeutic relevance, Nat Rev Cancer, 2005; 5(4):263-274). Studies show that T-cell receptor zeta subunit (TCR) is downregulated and Indoleamine 2,3-dioxygenase (IDO) is upregulated within the tumor draining lymph nodes as part of the elements involved in the regional immune suppression.

In addition to the suppressive effects medicated by infiltrating regulatory immune cells, tumor cells themselves can secret many molecules to actively inhibit cytotoxic T cell activation and function.

In some tumors, T cell intrinsic anergy and exhaustion is common, resulting from TCR ligation in the absence of engagement of co-stimulatory receptors on T cells such as CD28.

In the present disclosure, Component II of the combination therapy inhibits one or more immunosuppressive mechanisms and enhances a cancer specific immune response for eliminating tumor cells.

### Checkpoint Inhibitors

In some embodiments, Component II comprises a checkpoint inhibitor, such as an active agent that block the checkpoint pathway.

During adaptive immune response, activation of cytotoxic T cells is mediated by a primary signal between antigenic peptide/WIC molecule complexes on antigen presenting cells and the T cell receptor (TCR) on T cells. A secondary co-stimulatory signal is also important to active T cells. Antigen presentation in the absence of the secondary signal is not sufficient to activate T cells, for example CD4+T helper cells. The well-known co-stimulatory signal involves co-stimulatory receptor CD28 on T cells and its ligands B7-1/CD80 and B7-2/CD86 on antigen presenting cells (APCs). The B7-1/2 and CD28 interaction can augment antigen specific T cell proliferation and cytokine production. To tightly regulate an immune response, T cells also express CTLA-4 (anti-cytotoxic T-lymphocyte antigen 4), a co-inhibitory competitor of CD80 and CD86 mediated co-stimulation through the receptor CD28 on T cells, which can effectively inhibit T cell activation and function. CTLA-4 expression is often induced when CD28 interacts with B7-1/2 on the surface of an APC. CTLA-4 has higher binding affinity to the co-stimulatory ligand B7-1/2 (CD80/CD86) than the co-stimulatory receptor CD28, and therefore tips the balance from the T cell activating interaction between CD28 and B7-1/2 to inhibitory signaling between CTLA-4 and B7-1/2, leading to suppression of T cell activation. CTLA-4 upregulation is predominantly during the initial activation of T cells in the lymph node.

Antibodies that specifically bind to CTLA-4 have been used to inhibit this inhibitory checkpoint. The anti CTLA-4 IgG1 humanized antibody: ipilimumab binds to CTLA-4 and prevents the inhibition of CD28/B7 stimulatory signaling. They can lower the threshold for activation of T cells in lymphoid organs, also can deplete T regulatory cells within the tumor microenvironment (Simpson et al., Fc-dependent depletion of tumor-infiltrating regulatory T cells co-defines the efficacy of anti-CTLA-4 therapy against melanoma. J Exp. Med., 2013, 210: 1695-1710). Ipilimumab was recently approved by the U.S. Food and Drug Administration for the treatment of patients with metastatic melanoma.

In some embodiments, Component II of the combination therapy of the present disclosure may comprise an antagonist agent against CTLA-4 such as an antibody, a functional fragment of the antibody, a polypeptide, or a functional fragment of the polypeptide, or a peptide, which can bind to CTLA-4 with high affinity and prevent the interaction of B7-1/2 (CD80/86) with CTLA-4. In one example. The CTLA-4 antagonist is an antagonistic antibody, or a functional fragment thereof. Suitable anti-CTLA-4 antagonistic antibody include, without limitation, anti-CTLA-4 antibodies, human anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and the antibodies disclosed in U.S. Pat. Nos. 8,748,815; 8,529,902; 8,318,916; 8,017,114; 7,744,875; 7,605,238; 7,465,446; 7,109,003; 7,132,281; 6,984,720; 6,682,736; 6,207,156; 5,977,318; and European Patent No. EP1212422B1; and U.S. Publication Nos. US 2002/0039581 and US 2002/086014; and Hurwitz et al., Proc. Natl. Acad. Sci. USA, 1998, 95(17):10067-10071; the contents of each of which are incorporated by reference herein in their entirety.

Additional anti-CTLA-4 antagonist agents include, but are not limited to, any inhibitors that are capable of disrupting the ability of CTLA-4 to bind to the ligands CD80/86.

The inhibitory checkpoint receptor PD-1 (programmed death-1) is expressed on activated T cells and can induce inhibition and apoptosis of T cells following ligation by programmed death ligands 1 and 2 (PD-L1, also known as B7-H1, CD274), and PD-L2 (also known as B7-DC, CD273), which are normally expressed on epithelial cells and endothelial cells and immune cells (e.g., DCs, macrophages and B cells). PD-1 modulates T cell function mainly during the effector phase in peripheral tissues including tumor tissues. PD-1 is expressed on B cells and myeloid cells, in addition to activated T cells. Many human tumor cells can express PD-L1 and hijack this regulatory function to evade immune recognition and destruction by cytotoxic T lymphocytes. Tumor-associated PD-L1 has been shown to induce apoptosis of effector T cells and is thought to contribute to immune evasion by cancers.

The PD-1/PD-L1 immune checkpoint appears to be involved in multiple tumor types, for example, melanoma. PD-L1 not only provides immune escape for tumor cells but also turns on the apoptosis switch on activated T cells. Therapies that block this interaction have demonstrated promising clinical activity in several tumor types.

Component II comprises an active agent that blocks the PD-1 pathway include antagonistic peptides/antibodies and soluble PD-L1/2 ligands. Non-limiting examples of such an active agent are listed in Table 1.

TABLE 1
Agents that block the PD-1 and PD-L1/2 checkpoint pathway

| Agent | Description | Target |
|---|---|---|
| Nivolumab | Human IgG | PD-1 |
| (BMS-936558, ONO-4538, | | |
| MDX-1106 | | |
| Pembrolizumab | Humanized IgG4 | PD-1 |
| (MK-3475, lambrolizumab) | | |
| Pidilizumab (CT-011) | Humanized anti-PD-1 | PD-1 |
| | IgG1kappa | |
| AMP-224 | B7-DC/IgG1 fusion | PD-1 |
| | protein | |
| MSB0010718 (EMD-Serono) | Human IgG1 | PD-L1 |
| MEDI4736 | Engineered human | PD-L1 |
| | IgG 1kappa | |
| MPDL3280A | Engineered IgG1 | PD-L1 |
| AUNP-12 | branched 29-amino | PD-1 |
| | acid peptide | |

In accordance with the present disclosure, Component II comprises an antagonist agent against PD-1 and PD-L1/2 inhibitory checkpoint pathway. In one embodiment, the antagonist agent may be an antagonistic antibody that specifically binds to PD-1 or PD-L1/L2 with high affinity, or a functional fragment thereof. The PD-1 antibodies may be antibodies taught in U.S. Pat. Nos. 8,779,105; 8,168,757; 8,008,449; 7,488,802; 6,808,710; and PCT publication No.: WO 2012/145493; the contents of which are incorporated by references herein in their entirety. Antibodies that can specifically bind to PD-L1 with high affinity may be those disclosed in U.S. Pat. Nos. 8,552,154; 8,217,149; 7,943,743; 7,635,757; U.S. Publication No. 2009/0317368, and PCT Publication Nos. WO 2011/066389 and WO 2012/145493; the contents of which are incorporated herein by references in their entirety. In some examples, Component II comprises an antibody selected from 17D8, 2D3, 4H1, 5C4 (also known as nivolumab or BMS-936558), 4A11, 7D3 and 5F4 disclosed in U.S. Pat. No. 8,008,449; AMP-224, Pidilizumab (CT-011), and Pembrolizumab. In other examples, the anti-PD-1 antibody may be a variant of a human monoclonal anti-PD-1 antibody, for example a “mixed and matched” antibody variant in which a V_{H} sequence from a particular V_{H}/V_{L} pairing is replaced with a structurally similar V_{H} sequence, or a V_{L} sequence from a particular V_{H}/V_{L} pairing is replaced with a structurally similar V_{L} sequence, as disclosed in US publication No.: 2015/125463; the contents of which are incorporated by reference herein in its entirety.

In some embodiments, Component II comprises an antagonistic antibody that binds to PD-L1 with high affinity and disrupts the interaction between PD-1/PD-L1/2. Such antibodies may include, without limitation, 3G10, 12A4 (also referred to as BMS-936559), 10A5, 5F8, 10H10, 1B12, 7H1, 11E6, 12B7, and 13G4 disclosed in U.S. Pat. No. 7,943,743 (the contents of which are incorporated by reference in its entirety), MPDL3280A, MEDI4736, and MSB0010718. In another example, the anti-PD-L1 antibody may be a variant of a human monoclonal anti-PD-L1 antibody, for example a “mixed and matched” antibody variant in which a V_{H} sequence from a particular V_{H}/V_{L} pairing is replaced with a structurally similar V_{H} sequence, or a V_{L} sequence from a particular V_{H}/V_{L} pairing is replaced with a structurally similar V_{L} sequence, as disclosed in US publication No.: 2015/125463; the contents of which are incorporated by reference in its entirety.

In some embodiments, Component II comprises an antagonistic antibody that binds to PD-L2 with high affinity and disrupts the interaction between PD-1/PD-L1/2. Exemplary anti-PD-L2 antibodies may include, without limitation, antibodies taught by Rozali et al (Rozali et al., Programmed Death Ligand 2 in Cancer-Induced Immune Suppression, Clinical and Developmental Immunology, 2012, Volume 2012 (2012), Article ID 656340), and human anti-PD-L2 antibodies disclosed in U.S. Pat. No. 8,552,154 (the contents of which are incorporated herein by reference in their entirety).

In some embodiments, Component II comprises compounds that inhibit immunosuppressive signal induced due to PD-1, PD-L1 and/or PD-L2 such as cyclic peptidomimetic compounds disclosed in U.S. Pat. No. 9,233,940 to Sasikumar et al. (Aurigene Discovery Tech.), WO2015033303 to Sasikumar et al.; immunomodulating peptidomimetic compounds disclosed in WO2015036927 to Sasikumar et al.; 1,2,4-oxadiazole derivatives disclosed in US2015007302 to Govindan et al.; 1,3,4-oxadiazole and 1,3,4-thiadiazole compounds disclosed in WO2015033301 to Sasikumar et al.; or therapeutic immunomodulating compounds and derivatives or pharmaceutical salts of a peptide derivative of formula (I) or a stereoisomer of a peptide derivative of formula (I) disclosed in WO2015044900 to Sasikumar et al., the contents of each of which are incorporated herein by reference in their entirety.

In other embodiments, Component II comprises an antibody having binding affinity to both PD-L1 and PD-L2 ligands, for example the single agent of anti-PD-L1 and PD-L2 antibodies disclosed in PCT publication No.: WO2014/022758; the contents of which are incorporated by reference in its entirety.

In some embodiments, Component II comprises two or more antibodies selected from anti-PD-1 antibodies, PD-L1 antibodies and PD-L2 antibodies. In one example, an anti-PD-L1 antibody and an anti-PD-L2 antibody may be included in a single conjugate through a linker.

In some embodiments, Component II comprises a modulatory agent that can simultaneously block the PD-1 and PD-L1/2 mediated negative signal transduction. This modulatory agent may be a non-antibody agent. In some aspects, the non-antibody agents may be PD-L1 proteins, soluble PD-L1 fragments, variants and fusion proteins thereof. The non-antibody agents may be PD-L2 proteins, soluble PD-L2 fragments, variants and fusion proteins thereof. PD-L1 and PD-L2 polypeptides, fusion proteins, and soluble fragments can inhibit or reduce the inhibitory signal transduction that occurs through PD-1 in T cells by preventing endogenous ligands (i.e. endogenous PD-L1 and PD-L2) of PD-1 from interacting with PD-1. Additionally, the non-antibody agent may be soluble PD-1 fragments, PD-1 fusion proteins which bind to ligands of PD-1 and prevent binding to the endogenous PD-1 receptor on T cells. In one example, the PD-L2 fusion protein is B7-DC-Ig and the PD-1 fusion protein is PD-1-Ig. In another example, the PD-L1, PD-L2 soluble fragments are the extracellular domains of PD-L1 and PD-L2, respectively. In one embodiment, Component II comprises a non-antibody agent disclosed in US publication No.: 2013/017199; the contents of which are incorporated by reference herein in its entirety.

In addition to CTLA-4 and PD-1, other known immune inhibitory checkpoints include TIM-3 (T cell immunoglobulin and mucin domain-containing molecule 3), LAG-3 (lymphocyte activation gene-3, also known as CD223), BTLA (B and T lymphocyte attenuator), CD200R, KRLG-1, 2B4 (CD244), CD160, KIR (killer immunoglobulin receptor), TIGIT (T-cell immune-receptor with immunoglobulin and ITIM domains), VISTA (V-domain immunoglobulin suppressor of T-cell activation) and A2aR (A2a adenosine receptor) (Ngiow et al., Prospects for TIM3 targeted antitumor immunotherapy, Cancer Res., 2011, 71(21): 6567-6571; Liu et al., Immune-checkpoint proteins VISTA and PD-1 nonredundantly regulate murine T-cell responses, PNAS, 2015, 112(21): 6682-6687; and Baitsch et al., Extended Co-Expression of Inhibitory Receptors by Human CD8 T-Cells Depending on Differentiation, Antigen-Specificity and Anatomical Localization. 2012, Plos One, 7(2): e30852). These molecules that similarly regulate T-cell activation are being assessed as targets of cancer immunotherapy.

TIM-3 is a transmembrane protein constitutively expressed on IFN-γ-secreting T-helper 1 (Th1/Tc1) cells (Monney et al., Th1-specific cell surface protein Tim-3 regulates macrophage activation and severity of an autoimmune disease. Nature. 2002, 415:536-541), DCs, monocytes, CD8⁺ T cells, and other lymphocyte subsets as well. TIM-3 is an inhibitory molecule that down-regulates effector Th1/Tc1 cell responses and induces cell death in Th1 cells by binding to its ligand Galectin-9, and also induces peripheral tolerance (Fourcade et al. Upregulation of Tim-3 and PD-1 expression is associated with tumor antigen-specific CD8+ T cell dysfunction in melanoma patients. J experimental medicine. 2010; 207:2175-2186). Blocking TIM-3 can enhance cancer vaccine efficacy (Lee et al., The inhibition of the T cell immunoglobulin and mucin domain 3(Tim-3) pathway enhances the efficacy of tumor vaccine. Biochem. Biophys. Res Commun, 2010, 402: 88-93).

It has been shown that extracellular adenosine generated from hypoxia in the tumor microenvironment binds to A2a receptor which is expressed on a variety of immune cells and endothelial cells. The activation of A2aR on immune cells induces increased production of immunosuppressive cytokines (e.g., TGF-β, IL-10), upregulation of alternate immune checkpoint pathway receptors (e.g., PD-1, LAG-3), increased FOXP3 expression in CD4+ T cells driving a regulatory T cell phenotype, and induction of effector T cell anergy. Beavis et al demonstrated that A2aR blockade can improve effector T cell function and suppress metastasis (Beavis et al., Blockade of A2A receptors potently suppresses the metastasis of CD73+ tumors. Proc Natl Acad Sci USA, 2013, 110: 14711-14716). Some A2aR inhibitors are used to block A2aR inhibitory signal, including, without limitation, SCH58261, SYN115, ZM241365 and FSPTP (Leone et al., A2aR antagonists: Next generation checkpoint blockade for cancer immunotherapy, Comput Struct Biotechnol. J2015, 13: 265-272).

LAG-3 is a type I transmembrane protein expressed on activated CD4⁺ and CD8⁺ T cells, a subset of γδ T cells, NK cells and regulatory T cells (Tregs), and can negatively regulate immune response (Jha et al., Lymphocyte Activation Gene-3 (LAG-3) Negatively Regulates Environmentally-Induced Autoimmunity, PLos One, 2014, 9(8): e104484). LAG-3 negatively regulates T-cell expansion by inhibiting T cell receptor-induced calcium fluxes, thus controlling the size of the memory T-cell pool. LAG-3 signaling is important for CD4⁺ regulatory T-cell suppression of autoimmune responses, and LAG-3 maintains tolerance to self and tumor antigens via direct effects on CD8⁺ T cells. A recent study showed that blockade of both PD-1 and LAG-3 could provoke immune cell activation in a mouse model of autoimmunity, supporting that LAG-3 may be another important potential target for checkpoint blockade.

BTLA, a member of the Ig superfamily, binds to HVEM (herpesvirus entry mediator; also known as TNFRSF14 or CD270), a member of the tumor necrosis factor receptor superfamily (TNFRSF) (Watanabe et al., BTLA is a lymphocyte inhibitory receptor with similarities to CTLA-4 and PD-1 Nat Immunol, 2003, 4670-679. HVEM is expressed on T cells (e.g. CD8+ T cells). The HVEM-BTLA pathway plays an inhibitory role in regulating T cell proliferation (Wang et al., The role of herpesvirus entry mediator as a negative regulator of T cell-mediated responses, J Clin Invest., 2005, 115: 74-77). CD160 is another ligand of HVEM. The co-inhibitory signal of CD160/HVEM can inhibit the activation of CD4+ helper T cell (Cai et al., CD160 inhibits activation of human CD4⁺ T cells through interaction with herpesvirus entry mediator. Nat Immunol. 2008; 9:176-185).

CD200R is a receptor of CD200 that is expressed on myeloid cells. CD200 (OX2) is a highly expressed membrane glycoprotein on many cells. Studies indicated that CD200 and CD200R interaction can expand the myeloid-derived suppressor cell (MDSC) population (Holmannova et al., CD200/CD200R paired potent inhibitory molecules regulating immune and inflammatory responses; Part I: CD200/CD200R structure, activation, and function. Acta Medica (Hradec Kralove) 2012, 55(1):12-17; and Gorczynski, CD200 and its receptors as targets of immunoregulation, Curr Opin Investig Drug, 2005, 6(5): 483-488).

TIGIT is a co-inhibitory receptor that is highly expressed tumor-infiltrating T cells. In the tumor microenvironment, TIGIT can interact with CD226, a costimulatory molecule on T cells in cis, therefore disrupt CD226 dimerization. This inhibitory effect can critically limit antitumor and other CD8+ T cell-dependent responses (Johnston et al., The immunoreceptor TIGIT regulates antitumor and antiviral CD8(+) T cell effector function, Cancer cell, 2014, 26(6):923-937).

KIRs are a family of cell surface proteins expressed on natural killer cells (NKs). They regulate the killing function of these cells by interacting with MHC class I molecules expressed on any cell types, allowing the detection of virally infected cells or tumor cells. Most KIRs are inhibitory, meaning that their recognition of MEW molecules suppresses the cytotoxic activity of their NK cell (Ivarsson et al., Activating killer cell Ig-like receptor in health and disease, Frontier in Immu., 2014, 5: 1-9).

Additional coinhibitory signals that affect T cell activation include, but are not limited to KLRG-1, 2B4 (also called CD244), and VISTA (Lines et al., VISTA is a novel broad-spectrum negative checkpoint regulator for cancer immunotherapy, Cancer Immunol Res., 2014, 2(6): 510-517).

In accordance with the present disclosure, Component II comprises an antagonist or inhibitor of a co-inhibitory molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA, A2aR and other immune checkpoints. In some aspects, the antagonist agent may be an antagonistic antibody, or a functional fragment thereof, against a coinhibitory checkpoint molecule selected from CTLA-4, PD-1, PD-L1, PD-L2, TIM-3, LAG-3(CD223), BTLA, CD160, CD200R, TIGIT, KRLG-1, KIR, 2B4 (CD244), VISTA and A2aR.

In some embodiments, Component II comprises an antagonistic antibody, and/or a functional fragment thereof, specific to LAG-3(CD223). Such antagonistic antibodies can specifically bind to LAG-3(CD223) and inhibit regulatory T cells in tumors. In one example, it may be an antagonistic anti-LAG-3(CD223) antibody disclosed in U.S. Pat. Nos. 9,005,629 and 8,551,481. Component II may also comprise any inhibitor that binds to the amino acid motif KIEELE in the LAG-3(CD223) cytoplasmic domain which is essential for CD223 function, as identified using the methods disclosed in U.S. Pat. Nos. 9,005,629 and 8,551,481; the contents each of which are incorporated herein by reference in their entirety. Other antagonistic antibodies specific to LAG-3(CD223) may include antibodies disclosed in US publication NO. 20130052642; the contents of which is incorporated herein by reference in its entirety.

In some embodiments, Component II comprises an antagonistic antibody, and/or a functional fragment thereof, specific to TIM-3. Such antagonistic antibodies specifically bind to TIM-3 and can be internalized into TIM-3 expressed cells such as tumor cells to kill tumor cells. In other aspects, TIM-3 specific antibodies that specifically bind to the extracellular domain of TIM-3 can inhibit proliferation of TIM-3 expressing cells upon binding, e.g., compared to proliferation in the absence of the antibody and promote T-cell activation, effector function, or trafficking to a tumor site. In one example, the antagonistic anti-TIM-3 antibody may be selected from any antibody disclosed in U.S. Pat. Nos. 8,841,418; 8,709,412; 8,697,069; 8,647,623; 8,586,038; and 8,552,156; the contents of each of which are incorporated herein by reference in their entirety.

In addition, the antagonistic TIM-3 specific antibody may be monoclonal antibodies 8B.2C12, 25F.1D6 as disclosed in U.S. Pat. Nos. 8,697,069; 8,101,176; and 7, 470, 428; the contents of each of which are incorporated herein by reference in their entirety.

In other embodiments, Component II comprises an agent that can specifically bind to galectin-9 and neutralize its binding to TIM-3, including neutralizing antibodies disclosed in PCT publication NO. 2015/013389; the contents of which are incorporated by reference in its entirety.

In some embodiments, Component II comprises an antagonistic antibody, and/or a functional fragment thereof, specific to BTLA, including but not limited to antibodies and antigen binding portion of antibodies disclosed in U.S. Pat. Nos. 8,247,537; 8,580,259; fully human monoclonal antibodies in U.S. Pat. No. 8,563,694; and BTLA blocking antibodies in U.S. Pat. No. 8,188,232; the contents of each of which are incorporated herein by reference in their entirety.

Other additional antagonist agents that can inhibit BTLA and its receptor HVEM may include agents disclosed in PCT publication Nos.: 2014/184360; 2014/183885; 2010/006071 and 2007/010692; the contents of each of which are incorporated herein by reference in their entirety.

In certain embodiments, Component II comprises an antagonistic antibody, and/or or a functional fragment thereof, specific to KIR, for example IPH2101 taught by Benson et al., (A phase I trial of the anti-KIR antibody IPH2101 and lenalidomide in patients with relapsed/refractory multiple myeloma, Clin Cancer Res., 2015, May 21. pii: clincanres.0304.2015); the contents of which are incorporated by reference in its entirety.

In other embodiments, the antagonist agent may be any compound that can inhibit the inhibitory function of a coinhibitory checkpoint molecule selected from CTLA-4, PD-1, PD-L1, PD-L2, TIM-3, LAG-3(CD223), BTLA, CD160, CD200R, TIGIT KRLG-1, KIR, 2B4 (CD244), VISTA and A2aR.

In some examples, the antagonist agent may be a non-antibody inhibitor such as LAG-3-Ig fusion protein (IMP321) (Romano et al., J transl. Medicine, 2014, 12:97), and herpes simplex virus (HSV)-1 glycoprotein D (gD), an antagonist of BTLA)/CD160-HVEM) pathways (Lasaro et al., Mol Ther. 2011; 19(9): 1727-1736).

In some embodiments, Component II comprises an agent that is bispecific or multiple specific. As used herein, the terms “bispecific agent” and “multiple specific agent” refer to any agent that can bind to two targets or multiple targets simultaneously. In some aspects, the bispecific agent may be a bispecific peptide agent that has a first peptide sequence that binds a first target and a second peptide sequence that binds a second different target. The two different targets may be two different inhibitory checkpoint molecules selected from CTLA-4, PD-1 PD-L1, PD-L2, TIM-3, LAG-3(CD223), BTLA, CD160, CD200R, TIGIT, KRLG-1, KIR, 2B4 (CD244), VISTA and A2aR. A non-limiting example of bispecific peptide agents is a bispecific antibody or antigen-binding fragment thereof. Similarly, a multiple specific agent may be a multiple peptide specific agent that has more than one specific binding sequence domain for binding to more than one target. For example, a multiple specific polypeptide can bind at least two, at least three, at least four, at least five, at least six, or more targets. A non-limiting example of multiple-specific peptide agents is a multiple-specific antibody or antigen-binding fragment thereof.

In one example, such bispecific agent is the bispecific polypeptide antibody variants for targeting TIM-3 and PD-1, as disclosed in US publication No.: 2013/0156774; the content of which is incorporated herein by reference in its entirety.

In some embodiments, Component II comprises a conjugate that has one, two or multiple checkpoint antagonists/inhibitors connected via linkers in one conjugate.

In some embodiments, Component II comprises any agent that binds to and inhibits a checkpoint receptor. The checkpoint receptor is selected from the group consisting of CTLA-4, PD-1, CD28, inducible T cell co-stimulator (ICOS), B and T lymphocyte attenuator (BTLA), killer cell immunoglobulinlike receptor (KIR), lymphocyte activation gene 3 (LAG3), CD137, OX40, CD27, CD40L, T cell membrane protein 3 (TIM3), and adenosine A2a receptor (A2aR).

In one example, Component II comprises a CTLA-4 antagonist.

In another example, Component II comprises a PD-1 antagonist.

In yet another example, Component II comprises a PD-L1 antagonist.

### Radiation Therapies

In some embodiments, Component II comprises an agent for radiation therapy. Component I may be used to sensitive patients to radiation therapy and may be administered before and/or concurrent with the radiation therapy.

In some embodiments, Component II may comprise a radioactive agent. In one example, Component II comprises lutetium 177 (Lu 177) and dotatate, such as Lutathera. Component I may be given before and/or concurrent with Lutathera treatment. Component I may also be given after Lutathera treatment. Component I may be Conjugate 1.

### HDAC Inhibitors

Histone acetylation is an important regulatory mechanism of transcription of genes and can cause an increase or decrease in gene transcription: acetylation of histones causes decondensation of chromatin; deactylation causes condensation of chromatin; acetylation can occur on histones, DNA binding proteins, receptors, DNA repair enzymes, transcriptional coregulators, and DNA binding transcription factors.

Most Histone deacetylases (HDACs) exist in large complexes with multiple transcriptional co-repressors and contribute to gene silencing via specific targeting by repressors and the constitutive association with histones. Class I HDACs include HDAC1, HDAC2, HDAC3 and HDAC8. HDAC1 is overexpressed in prostate, gastric, lung, esophageal, colon, and breast cancers. HDAC2 is overexpressed in colorectal, cervical, and gastric cancers. HDAC3 is overexpressed in colon and breast cancers. HDAC8 is overexpressed in neuroblastoma. Class IIa HDACs include HDAC4, HDAC5, HDAC7 and HDAC9. Class IIb HDACs include HDAC6 (overexpressed in mammary tumors) and HDAC10. Class III includes NAD dependent homologs 1-7 of the yeast Sir2 proteins. Class IV includes HDAC11 overexpressed in rhabdomyosarcoma. HDAC inhibitors have been developed to treat cancers, such as neuroendocrine tumors (NETs), prostate cancer, gastric cancer, lung cancer, esophageal cancer, colon cancer, colorectal cancer, cervical cancer, and breast cancer. They may induce cancer cell cycle arrest, differentiation and cell death, reduce angiogenesis, and modulate immune responses.

In some embodiments, Component II may be any HDAC inhibitor which inhibits the functions of any HDAC enzyme. Any HDAC inhibitor disclosed in Table 1 of Eckschlager et al., International Journal of Molecular Sciences, vol. 18(7):1414 (2017), the contents of which are incorporated herein by reference in their entirety. Non-limiting examples of HDAC inhibitors include benzamides such as Entinostat, Tacedinaline, 4SC202, or Mocetinostat; cyclic tetrapeptides such as Romidepsin; hydroxamic acids such as Trichostatin A, SAHA, Belinostat, Panabiostat, Givinostat, Resminostat, Abexinostat, Quisinostat, Rocilinostat, Practinostat, or CHR-3996; sirtuins inhibitors such as Nicotinamide, Sirtinol, Cambinol, or EX-527; or short chain fatty acids such as Valproic acid, Butyric acid, or Phenylbutyric acid. In some embodiments, the HDAC inhibitor is vorinostat (a kind of suberanilohydroxamic acid or SAHA).

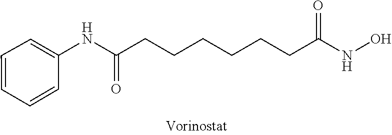 [SMILES: O=C(CCCCCCC(=O)Nc1ccccc1)NO]

HSP90 Inhibitors and HSP90-binding Conjugates

Inhibition of heat shock protein 90 (Hsp90) results in degradation of its client proteins via the ubiquitin proteasome pathway. Unlike other chaperone proteins, the client proteins of Hsp90 are mostly protein kinases or transcription factors involved in signal transduction, and a number of its client proteins have been shown to be involved in the progression of cancer. Hsp90 has been shown by mutational analysis to be necessary for the survival of normal eukaryotic cells. However, Hsp90 is overexpressed in many tumor types, indicating that it may play a significant role in the survival of cancer cells and that cancer cells may be more sensitive to inhibition of Hsp90 than normal cells.

In some embodiments, Component II is an HSP90 inhibitor. Non-limiting examples include ganetespib, geldanamycin (tanespimycin), IPI-493, macbecins, tripterins, tanespimycins, 17-AAG (alvespimycin), KF-55823, radicicols, KF-58333, KF-58332, 17-DMAG, IPI-504, BIIB-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, celastrol, or a tautomer, analog, or derivative thereof.

In some embodiments, Component II is a conjugate comprising an active agent or prodrug thereof attached to a targeting moiety, wherein the targeting moiety binds to a heat shock protein, such as HSP90. The targeting moiety may be selected from ganetespib, geldanamycin (tanespimycin), IPI-493, macbecins, tripterins, tanespimycins, 17-AAG (alvespimycin), KF-55823, radicicols, KF-58333, KF-58332, 17-DMAG, IPI-504, BIIB-021, BIIB-028, PU-H64, PU-H71, PU-DZ8, PU-HZ151, SNX-2112, SNX-2321, SNX-5422, SNX-7081, SNX-8891, SNX-0723, SAR-567530, ABI-287, ABI-328, AT-13387, NSC-113497, PF-3823863, PF-4470296, EC-102, EC-154, ARQ-250-RP, BC-274, VER-50589, KW-2478, BHI-001, AUY-922, EMD-614684, EMD-683671, XL-888, VER-51047, KOS-2484, KOS-2539, CUDC-305, MPC-3100, CH-5164840, PU-DZ13, PU-HZ151, PU-DZ13, VER-82576, VER-82160, VER-82576, VER-82160, NXD-30001, NVP-HSP990, SST-0201CL1, SST-0115AA1, SST-0221AA1, SST-0223AA1, novobiocin, herbinmycin A, radicicol, CCT018059, PU-H71, celastrol, or a tautomer, analog, or derivative thereof.

In some examples, Component II comprises SN-38 or irinotecan, lenalidomide, vorinostat, 5-Fluorouracil (5-FU), abiraterone, bendamustine, crizotinib, doxorubicin, pemetrexed, fulvestrant, topotecan, Vascular Disrupting Agent (VDA), or a fragment, derivative, or analog thereof as an active agent.

In some examples, Component II may be any conjugate of PCT Application No. PCT/US13/36783 (WO2013/158644) filed on Apr. 16, 2013, the contents of which are incorporated herein by reference.

In one example, Component II is Conjugate 2 having a structure of

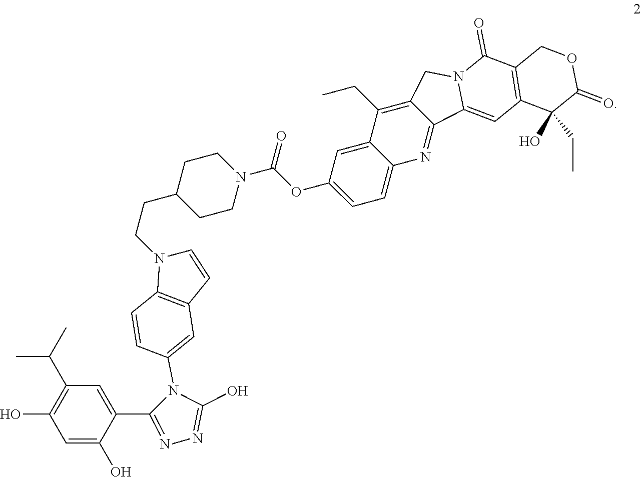 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]

((S)-4,11-diethyl-4-hydroxy-3,14-dioxo-3,4,12,14-tetrahydro-1H-pyrano[3′,4′:6,7]indolizino[1,2-b]quinolin-9-yl 4-(2-(5-(3-(2,4-dihydroxy-5-isopropylphenyl)-5-hydroxy-4H-1,2,4-triazol-4-yl)-1H-indol-1-yl)ethyl)piperidine-1-carboxylate) or its tautomer.

Conjugate 2 is an injectable, synthetic small molecule drug conjugate comprised of ganetespib attached through a cleavable linker to SN-38, the active metabolite of the marketed topoisomerase I inhibitor, irinotecan. This conjugate leverages the enhanced tumor targeting and preferential tumor retention properties of HSP90 to deliver SN-38 resulting in broad preclinical antitumor activity.

### Formulations and Administration

Each component in the combination therapy of the present disclosure can be formulated using one or more pharmaceutically acceptable excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Component I and Component II can each be administered in different compositions.

Non-limiting examples of the excipients include any and all solvents, dispersion media, diluents, or other liquid vehicles, dispersion or suspension aids, surface active agents, isotonic agents, thickening or emulsifying agents, and preservatives. Excipients may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of each component may include one or more excipients, each in an amount that together increases the stability of the active agents.

Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of the present disclosure.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

In some embodiments, Conjugate 1 is administered to the patient in a pharmaceutical composition, wherein the pharmaceutical composition has a pH of about 4.0 to about 5.0. In some embodiments, the pharmaceutical composition comprises acetate buffer (sodium acetate and acetic acid) having a pH of about 4.0 to about 4.8. In some embodiments, the pharmaceutical composition further comprises mannitol and polyoxyl 15 hydroxystearate.

In one embodiment, a composition for solution for injection is provided for administering Conjugate 1. The solution comprises Conjugate 1, mannitol, Polyoxyl 15 Hydroxystearate, and aqueous acetate buffer. The composition may be infused intravenously (IV).

In some embodiments, Conjugate 2 is administered to the patient in a pharmaceutical composition, wherein the pharmaceutical composition has a pH of above 8.0, or above 9.0, or above 10.0. In some embodiments, the pharmaceutical composition comprises sodium chloride solution. The composition may be administered intravenously (IV). Conjugate 2 may be in its lactone form or a carboxylic acid salt form.

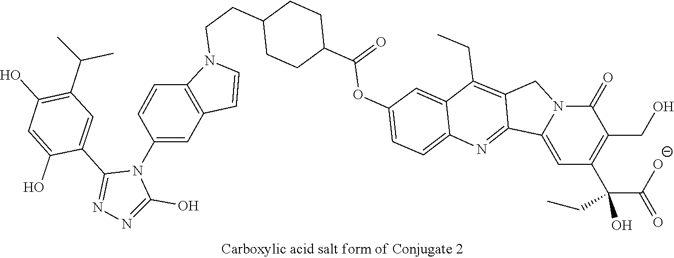 [SMILES: CCc1c2c(nc3ccc(OC(=O)C4CCC(CCn5ccc6cc(-n7c(O)nnc7-c7cc(C(C)C)c(O)cc7O)ccc65)CC4)cc13)-c1cc([C@@](O)(CC)C(=O)[O-])c(CO)c(=O)n1C2]

### Particles

In some embodiments, at least one component of the combination therapy is formulated in particles, such as polymeric particles, lipid particles, inorganic particles, or combinations thereof (e.g., lipid stabilized polymeric particles). In some embodiments, the particles are solid polymeric particles or contain a polymeric matrix. The particles can contain any of the polymers described herein or derivatives or copolymers thereof. The particles generally contain one or more biocompatible polymers. The polymers can be biodegradable polymers. The polymers can be hydrophobic polymers, hydrophilic polymers, or amphiphilic polymers. In some embodiments, the particles contain one or more polymers having an additional targeting moiety attached thereto.

The component of the combination therapy may be formulated with any particle disclosed in WO2014/106208 to Bilodeau et al. filed Dec. 30, 2013, the contents of which are incorporated herein by reference in their entirety.

The size of the particles can be adjusted for the intended application. The particles can be nanoparticles or microparticles. The particle can have a diameter of about 10 nm to about 10 microns, about 10 nm to about 1 micron, about 10 nm to about 500 nm, about 20 nm to about 500 nm, or about 25 nm to about 250 nm. In some embodiments, the particle is a nanoparticle having a diameter from about 25 nm to about 250 nm. It is understood by those in the art that a plurality of particles will have a range of sizes and the diameter is understood to be the median diameter of the particle size distribution.

In some embodiments, the weight percentage of the component of the combination therapy in the particles is at least about 0.05%, 0.1%, 0.5%, 1%, 5%, 10%, 15%, 20%, 25%, 30%, 35%, 40%, 45%, or 50% such that the sum of the weight percentages of the components of the particles is 100%. In some embodiments, the weight percentage of the component in the particles is from about 0.5% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the weight percentages of all the components of the particles is 100%.

### Administration

The components of the combination therapy may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them to cross the blood-brain barrier, vascular barrier, or other epithelial barrier.

The formulations described herein contain an effective amount of the components in a pharmaceutical carrier appropriate for administration to a patient in need thereof. The formulations may be administered parenterally (e.g., by injection or infusion). The formulations or variations thereof may be administered in any manner including enterally, topically (e.g., to the eye), or via pulmonary administration. In some embodiments, the formulations are administered topically.

### Dosing

The exact amount required by the patient for each component will vary from subject to subject, depending on the species, age, and general condition of the subject, the severity of the disease, the particular composition, its mode of administration, its mode of activity, and the like.

Components of the combination therapy are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, the components of the combination therapy in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect.

The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

The concentration of the components may be between about 0.01 mg/mL to about 50 mg/mL, about 0.1 mg/mL to about 25 mg/mL, about 0.5 mg/mL to about 10 mg/mL, or about 1 mg/mL to about 5 mg/mL in the pharmaceutical composition.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the monomaleimide compounds of the present invention are C to a subject in split doses. The monomaleimide compounds may be formulated in buffer only or in a formulation described herein.

A subject may receive the combination therapy for any suitable length, such as a week, 2 weeks, 3 weeks, 4 weeks, a month, 2 months, 3 months, 4 months, 5 months, 6 months, 7 months, 8 months, 9 months, 10 months, 11 months, a year, or until a predetermined milestone is reached (e.g., a TGI % of above 90%, 95%, or 99%).

### II. Methods of Using the Combination Therapy

One aspect of the present disclosure provides methods for treating a subject having a hyperproliferative disorder such as cancer, wherein the method comprises a combination therapy of at least two distinct therapeutic agents. In some embodiments, the method comprises administering to the patient: (A) a first component which comprises, as an active agent, Compound I, or a prodrug, derivative, or pharmaceutically-acceptable salt thereof; and (B) a second component which comprises, as an active agent, Compound II, or a prodrug, derivative, or a pharmaceutically-acceptable salt thereof.

According to the present disclosure, cancer may be characterized by tumors, e.g., solid tumors or any neoplasm. In some embodiments, the cancer is a solid tumor. Large drug molecules have limited penetration in solid tumors. The penetration of large drug molecules is slow. On the other hand, small molecules such as small molecule conjugates may penetrate solid tumors rapidly and more deeply. Regarding penetration depth of the drugs, larger molecules penetrate less, despite having more durable pharmacokinetics.

In some embodiments, the combination therapy inhibits cancer and/or tumor growth. The combination therapy may also reduce, including cell proliferation, invasiveness, and/or metastasis, thereby rendering them useful for the treatment of a cancer.

In some embodiments, the combination therapy may be used to prevent the growth of a tumor or cancer, and/or to prevent the metastasis of a tumor or cancer. In some embodiments, the combination therapy may be used to shrink or destroy a cancer.

In some embodiments, the combination therapy is useful for inhibiting proliferation of a cancer cell. In some embodiments, the combination therapy is useful for inhibiting cellular proliferation, e.g., inhibiting the rate of cellular proliferation, preventing cellular proliferation, and/or inducing cell death. In general, the combination therapy can inhibit cellular proliferation of a cancer cell or both inhibiting proliferation and/or inducing cell death of a cancer cell. In some embodiments, cell proliferation is reduced by at least about 25%, about 50%, about 75%, or about 90% after treatment with the combination therapy of the present disclosure compared with cells with no treatment. In some embodiments, cell cycle arrest marker phospho histone H3 (PH3 or PHH3) is increased by at least about 50%, about 75%, about 100%, about 200%, about 400% or about 600% after treatment with combination therapy compared with cells with no treatment. In some embodiments, cell apoptosis marker cleaved caspase-3 (CC3) is increased by at least 50%, about 75%, about 100%, about 200%, about 400% or about 600% after treatment with combination therapy compared with cells with no treatment.

Furthermore, in some embodiments, combination therapy is effective for inhibiting tumor growth, whether measured as a net value of size (weight, surface area or volume) or as a rate over time, in multiple types of tumors.

In some embodiments, the size of a tumor is reduced by about 60% or more after treatment with the combination therapy. In some embodiments, the size of a tumor is reduced by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, by a measure of weight, and/or area and/or volume.

In some embodiments, tumor growth inhibition (TGI) of a subject receiving the combination therapy may be at least about 80%, 85%, 90%, 95%, or 99%.

The cancers treatable by combination therapy of the present disclosure generally occur in mammals. Mammals include, for example, humans, non-human primates, dogs, cats, rats, mice, rabbits, ferrets, guinea pigs, horses, pigs, sheep, goats, and cattle. In various embodiments, the cancer is lung cancer, breast cancer, e.g., mutant BRCA1 and/or mutant BRCA2 breast cancer, non-BRCA-associated breast cancer, colorectal cancer, ovarian cancer, pancreatic cancer, colorectal cancer, bladder cancer, prostate cancer, cervical cancer, renal cancer, leukemia, central nervous system cancers, myeloma, and melanoma.

In some embodiments, the cancer is a neuroendocrine cancer such as but not limited to small cell lung cancer (SCLC), adrenal medullary tumors (e.g., pheochromocytoma, neuroblastoma, ganglioneuroma, or paraganglioma), gastroenteropancreatic neuroendocrine tumors (e.g., carcinoids, gastrinoma, glucagonoma, vasoactive intestinal polypeptide-secreting tumor, pancreatic polypeptide-secreting tumor, or nonfunctioning gastroenteropancreatic tumors), meduallary thyroid cancer, Merkel cell tumor of the skin, pituitary adenoma, and pancreatic cancer. The somatostatin receptor SSTR2 is over expressed on 50-90% of neuroendocrine cancers. In some embodiments, the neuroendocrine cancer is a primary neuroendocrine cancer. In some embodiments, the neuroendocrine cancer is a neuroendocrine metastasis. Neuroendocrine metastatic may be in liver, lung, bone, or brain of a subject. In certain embodiments, the cancer is brain cancer, human lung carcinoma, ovarian cancer, pancreatic cancer or colorectal cancer.

In one embodiment, the combination therapy of the present disclosure is used to treat small cell lung cancer. About 12%-15% of patients having lung cancer have small cell lung cancer. Survival in metastatic small cell lung cancer is poor. Survival rate is below 5% five years after diagnosis. US incidence of small cell lung cancer is about 26K-30K. Among these patients, about 40%-80% are SSTR2 positive.

In some embodiments, the combination therapy of the present disclosure is used to treat patients with tumors that express or over-express the somatostatin receptor (SSTR positive). Such patients can be identified with any method known in the art, such as but not limited to using a radionuclide imaging agent, a radiolabeled somatostatin analog imaging agent, SSTR scintigraphy or SSTR positron emission tomography (PET). In one embodiment, ¹¹¹Indium (Indium111)-labeled pentetreotide scintigraphy (OctreoScan™) is used to identify patients with SSTR-expressing tumors. In another embodiment, a 68Ga conjugate such as 68Ga-DOTA-TATE, 68Ga-DOTA-TOC, or 68Ga-DOTA-NOC is used in PET imaging to identify patients with SSTR-expressing tumors. Patients who show positive scan results detected with Indium111-labeled pentetreotide scintigraphy are treated with the combination therapy of the present disclosure.

In some embodiments, the combination therapy of the present disclosure is used to treat patients with tumors that have low SSTR expression. A component of the combination therapy may be administered to the patient to increase SSTR expression as a pre-treatment.

The expression of SSTR can be measured with any suitable method known to a person of ordinary skill in the art. SSTR mRNA levels can be detected with Northern blotting, in-situ hybridization and reverse transcription-polymerase chain reaction (RT-PCR). SSTR protein levels can be measured with Western blotting and immunohistochemistry (IHC). For example, immunoreactive score (IRS), the human epidermal growth factor receptor 2 (HER2)/neu score, and/or the hormone receptor score (H score) of formalin-fixed, paraffin-embedded tumour samples (FFPE) may be obtained. Samples having values ≥3 IRS points, a HER2/neu score of 2+ or 3+, and/or an H score ≥50 points are generally considered SSTR positive. Samples having values below the above values are generally considered low in SSTR expression.

In one embodiment, the combination therapy of the present disclosure is used to treat patients having a histologically proven locally advanced or metastatic high grade neuroendocrine carcinoma (NEC). In some embodiments, the patients may have small cell and large cell neuroendocrine carcinoma of unknown primary or any extrapulmonary site. In some embodiments, the patients may have well differentiated G3 neuroendocrine neoplasms if Ki-67>30%. In some embodiments, the patients may have neuroendocrine prostate cancer (de novo or treatment-emergent) of prostate if small cell or large cell histology. In some embodiments, the patients may have mixed tumors, e.g. mixed adenoneuroendocrine carcinoma (MANEC) or mixed squamous or acinar cell NEC if the high grade (small or large cell) NEC component comprises >50% of the original sample or subsequent biopsy. In some embodiments, the patients may have castrate resistant prostate cancer (CRPC).

A feature of the components of the combination therapy is relatively low toxicity to an organism while maintaining efficacy at inhibiting, e.g. slowing or stopping tumor growth. As used herein, “toxicity” refers to the capacity of a substance or composition to be harmful or poisonous to a cell, tissue organism or cellular environment. Low toxicity refers to a reduced capacity of a substance or composition to be harmful or poisonous to a cell, tissue organism or cellular environment. Such reduced or low toxicity may be relative to a standard measure, relative to a treatment or relative to the absence of a treatment. For example, Conjugate 1, which comprises DM1 as an active agent, has a toxicity lower than DM1 administered alone. Conjugate 2, which comprises SN-38 as an active agent, has a toxicity lower than SN-38 administered alone.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaiase. Neutropenia, thrombopenia, white blood cell (WBC) count, complete blood cell (CBC) count may also be metrics of toxicity. Pharmacologic indicators of toxicity include elevated aminotransferases (AST/ALT) levels, neurotoxicity, kidney damage, GI damage and the like. In one embodiment, the combination therapy of the present disclosure do not cause a significant change of a subject's body weight. The body weight loss of a subject is less about 30%, about 20%, about 15%, about 10%, or about 5% after treatment with the combination therapy of the present disclosure. In another embodiment, the combination therapy of the present disclosure does not cause a significant increase of a subject's AST/ALT levels. The AST or ALT level of a subject is increased by less than about 30%, about 20%, about 15%, about 10%, or about 5% after treatment with the combination therapy of the present disclosure. In yet another embodiment, the combination therapy of the present disclosure does not cause a significant change of a subject's CBC or WBC count after treatment with the combination therapy of the present disclosure. The CBC or WBC level of a subject is decreased by less than about 30%, about 20%, about 15%, about 10%, or about 5% after treatment with the combination therapy of the present disclosure.

### III. Kits and Devices

One aspect of the present disclosure provides a variety of kits and devices for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for inhibiting tumor cell growth in vitro or in vivo, comprising at least two distinct therapeutic agents. In some embodiments, the kit for inhibiting tumor cell growth comprises: (A) a first component which comprises, as an active agent, Compound I, or a prodrug, derivative, or pharmaceutically-acceptable salt thereof; and (B) a second component which comprises, as an active agent, Compound II, or a prodrug, derivative, or a pharmaceutically-acceptable salt thereof.

The kit may further comprise packaging and instructions and/or a delivery agent to form a formulation composition. The delivery agent may comprise a saline, a buffered solution, or any delivery agent disclosed herein. The amount of each agent may be varied to enable consistent, reproducible higher concentration saline or simple buffer formulations. The agents may also be varied in order to increase the stability of the components of the combination therapy over a period of time and/or under a variety of conditions.

The present disclosure provides devices which may incorporate components of the combination therapy. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver components of the combination therapy according to single, multi- or split-dosing regiments. The devices may be employed to deliver components of the combination therapy across biological tissue, intradermal, subcutaneously, or intramuscularly.

### IV. Definitions

The term “compound”, as used herein, is meant to include all stereoisomers, geometric isomers, tautomers, and isotopes of the structures depicted. In the present application, compound is used interechangably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The terms “subject” or “patient”, as used herein, refer to any organism to which the combination therapy may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A “target”, as used herein, shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In still other embodiments, a target may refer to a molecular structure to which a targeting moiety or ligand binds, such as a hapten, epitope, receptor, dsDNA fragment, carbohydrate or enzyme. A target may be a type of tissue, e.g., neuronal tissue, intestinal tissue, pancreatic tissue, liver, kidney, prostate, ovary, lung, bone marrow, or breast tissue.

The “target cells” that may serve as the target for the combination therapy, are generally animal cells, e.g., mammalian cells. The present method may be used to modify cellular function of living cells in vitro, i.e., in cell culture, or in vivo, in which the cells form part of or otherwise exist in animal tissue. Thus, the target cells may include, for example, the blood, lymph tissue, cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject invention), dermal/epidermal cells, cells of the vagina and rectum, cells of internal organs including cells of the placenta and the so-called blood/brain barrier, etc. In general, a target cell expresses at least one type of SSTR. In some embodiments, a target cell can be a cell that expresses an SSTR and is targeted by a conjugate described herein, and is near a cell that is affected by release of the active agent of the conjugate. For example, a blood vessel expressing an SSTR that is in proximity to a tumor may be the target, while the active agent released at the site will affect the tumor.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease, disorder or condition in the enhancement of desirable physical or mental development and conditions in an animal, e.g., a human.

The term “modulation” is art-recognized and refers to up regulation (i.e., activation or stimulation), down regulation (i.e., inhibition or suppression) of a response, or the two in combination or apart. The modulation is generally compared to a baseline or reference that can be internal or external to the treated entity.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to affect a measurable improvement or prevention of at least one symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “prodrug” refers to an agent, including a small organic molecule, peptide, nucleic acid or protein, that is converted into a biologically active form in vitro and/or in vivo. Prodrugs can be useful because, in some situations, they may be easier to administer than the parent compound (the active compound). For example, a prodrug may be bioavailable by oral administration whereas the parent compound is not. The prodrug may also have improved solubility in pharmaceutical compositions compared to the parent drug. A prodrug may also be less toxic than the parent. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The terms “polypeptide,” “peptide” and “protein” generally refer to a polymer of amino acid residues. As used herein, the term also applies to amino acid polymers in which one or more amino acids are chemical analogs or modified derivatives of corresponding naturally-occurring amino acids or are unnatural amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general and unless otherwise specified, an analog of a particular nucleotide has the same base-pairing specificity; i.e., an analog of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. An antisense nucleic acid is a polynucleotide that interferes with the expression of a DNA and/or RNA sequence. The term nucleic acids refers to a string of at least two base-sugar-phosphate combinations. Natural nucleic acids have a phosphate backbone. Artificial nucleic acids may contain other types of backbones, but contain the same bases as natural nucleic acids. The term also includes PNAs (peptide nucleic acids), phosphorothioates, and other variants of the phosphate backbone of native nucleic acids.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable salt(s)” refers to salts of acidic or basic groups that may be present in compounds used in the present compositions. Compounds included in the present compositions that are basic in nature are capable of forming a variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

### EXAMPLES

### Example 1: Synthesis and HPLC Analysis of Conjugate 1 and Conjugate 2

In some embodiments, Component I is a conjugate comprising an active agent or prodrug thereof attached to a targeting moiety, wherein the targeting moiety binds to a somatostatin receptor (SSTR), such as SSTR2. Synthesis and HPLC analysis of the SSTR-targeting conjugates can be carried out with methods disclosed in the Examples A, 1-7, and 14 of PCT Application No. PCT/US15/38569 (WO2016/004048) filed on Jun. 30, 2015, the contents of which are incorporated herein by reference in their entirety. In particular, Conjugate 1 or a pharmaceutically-acceptable salt thereof can be prepared according to Example 14 of PCT/US15/38569.

In some embodiments, Component II is a conjugate comprising an active agent or prodrug thereof attached to a targeting moiety, wherein the targeting moiety binds to a heat shock protein, such as HSP90. Synthesis and HPLC analysis of the HSP90-targeting conjugates can be carried out with methods disclosed in Examples 1, 6, 8, 1-29 of PCT Application No. PCT/US13/36783 (WO2013/158644) filed on Apr. 16, 2013, the contents of which are incorporated herein by reference in their entirety. In particular, Conjugate 2 or a pharmaceutically-acceptable salt thereof can be prepared according to Example 6 of PCT/US13/36783.

### Example 2: Antitumor Efficacy of Conjugate 1 in Combination with Conjugate 2

The purpose of this study was to evaluate the antitumor efficacy of Conjugate 1 in combination with Conjugate 2 in the SSTR2 expressing NCI-H69 human SCLC xenograft model in nude mice (CrTac:NCr-Foxn1nu).

In this experiment, the compounds were administered iv once a week for four weeks, each group consisted of ten animals and treated groups included: Conjugate 1 at 0.7 mg/kg or 2.0 mg/kg, Conjugate 2 at 25 mg/kg, and the combination of Conjugate 2 at 25 mg/kg and Conjugate 1 at 0.7 mg/kg given with two different sequences: (a) Conjugate 1 24 h before Conjugate 2 (combo 1); and (b) Conjugate 2 24 h before Conjugate 1 (combo 2). Animals were assessed for body weight (BW) changes and their health was monitored. Tumor volumes and BW were measured on a 2× weekly schedule. Percent tumor growth inhibition (TGI) was determined on Day 25. Percent TGI was defined as the difference between the metabolic tumor volume (MTV) of the vehicle and the MTV of each treated group, expressed as a percentage of the MTV of the vehicle group at the end of study. Statistical significance was determined by one-way ANOVA using Graphpad software.

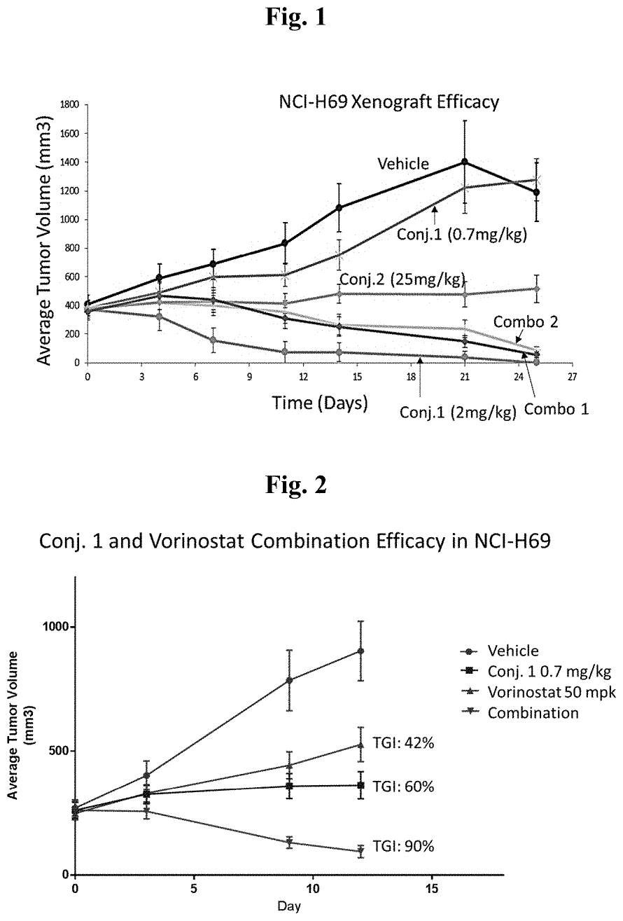

All groups experienced no mean group BW loss. On Day 25, Conjugate 1 at 0.7 mg/kg did not demonstrate statistically significant TGI compared to vehicle alone with −7% (P>0.999). However, Conjugate 1 at 2 mg/kg was significant with a TGI of 99% (P<0.0001) compared to vehicle. Conjugate 2 at 25 mg/kg did demonstrate statistically significant efficacy compared to vehicle alone with 57% TGI (P=0.0006). Both combination groups demonstrated statistically significant efficacy compared to vehicle control with 95% and 93% TGI for combo 1 and combo 2, respectively (P<0.0001). In addition, both combinations were superior to Conjugate 1 0.7 mg/kg treatment (P<0.0001) and Conjugate 2 treatment at 25 mg/kg (P<0.05). Data are summarized in FIG. 1. In summary, these data suggest that Conjugate 1 and Conjugate 2 can be combined for greater efficacy than monotherapy of each of them alone when given at the same dose level as in the combination therapies.

### Example 3: Evaluation of Immune Cell Populations Following Treatment with Conjugate 1

Evaluation of immune cell changes in Pan02 mouse model (pancreatic cancer syngeneic mouse model) is conducted. In this study, a broad look at immune cell changes is explored by broadly profiling immune cell changes in mice. For example, immune cell population is counted.

A biopsy is performed after treatment with Conjugate 1. Tumor infiltrating lymphocytes (TILs) dispersed in the stroma between the carcinoma cells are assessed independently by two trained histopathologists.

The expressions of immune checkpoint receptors on T cells and their cognate ligands on tumor-associated macrophages (TAMs) are analyzed. For example, CTLA-4, PD-1 expressions on CD4+ and CD8+ T cells upon Conjugate 1 treatment are analyzed.

Chemokines and cytokines in treated mice are also measured.

### Example 4: Antitumor Efficacy of Conjugate 1 in Combination with a Checkpoint Inhibitor

The purpose of this in vivo study is to evaluate the antitumor efficacy of Conjugate 1 in combination with a checkpoint inhibitor in the Pan02 mouse model of pancreatic cancer.

Mice are separated into the following groups: 1. Treatment with vehicle; 2. Treatment with Conjugate 1; 3. Treatment with a PD-1 blocking antibody; 4. Treatment with a PD-L1 antibody; 5. Treatment with Conjugate 1 and a PD-1 blocking antibody; and 6. Treatment with Conjugate 1 and a PD-L1 blocking antibody. The body weight (BW) and health of the mice are monitored. Tumor volumes are measured and tumor growth inhibitions are determined.

### Example 5. Antitumor Efficacy of Conjugate 1 in Combination with an HDAC Inhibitor

Antitumor efficacy of Conjugate 1 in combination with an HDAC inhibitor, vorinostat, was studied with a small cell lung cancer (SCLC) model such as NCI-H69.

Mice were separated into the following groups: 1. Treatment with vehicle; 2. Treatment with Conjugate 1 (0.7 mg/kg); 3. Treatment with vorinostat (50 mg/kg); and 4. Combination treatment with Conjugate 1 and vorinostat. In Group 4, mice were treated with Conjugate 1 once per week via IV for 2 weeks (a total of 2 doses) and vorinostat 5 days a week for 2 weeks via IP (a total of 10 doses).

The body weight (BW) and health of the mice were monitored. Tumor volumes were measured and tumor growth inhibitions were determined. As shown in FIG. 2, Conjugate 1 treatment yielded a tumor growth inhibition of 60% (P<0.0001). Vorinostat treatment yielded a tumor growth inhibition of 42% (P=0.003). The combo therapy yielded a tumor inhibition of 90%. Statistically significant (P=0.033) increase in tumor growth inhibition was seen with Conjugate 1 and Vorinostat combination in NCI-H69 xenograft model. 5 out of 10 tumors showed regressions with the combination treatment (<100 mm³).

Similar studies are carried out to measure antitumor efficacy of Conjugate 1 in combination with an HDAC inhibitor in a pheochromocytoma model such as PC12.

### Example 6. SSTR2 Expression in Tumor Tissues

The targeting peptide of Conjugate 1 selectively binds to SSTR2, triggers receptor internalization leading to the accumulation of the DM1 to provide antitumor activity by disrupting microtubule networks causing apoptosis and mitotic catastrophe. In preclinical studies, single agent Conjugate 1 treatment leads to complete and sustained tumor regression in xenograft models that over-express SSTR2.

This study was conducted to evaluate whether epigenetic modulation from HDAC or other epigenetic modulators would increase the expression of SSTR2; and whether the synergy of the DM1 payload and epigenetic modulators may allow for the broadening of the tumors treatable with Conjugate 1.

Mice bearing NCI-H69 tumors (small cell lung cancer) were treated with Vorinostat for 10 days IP (q.d.×5). Tumors were collected and processed for SSTR2 expression by IHC.

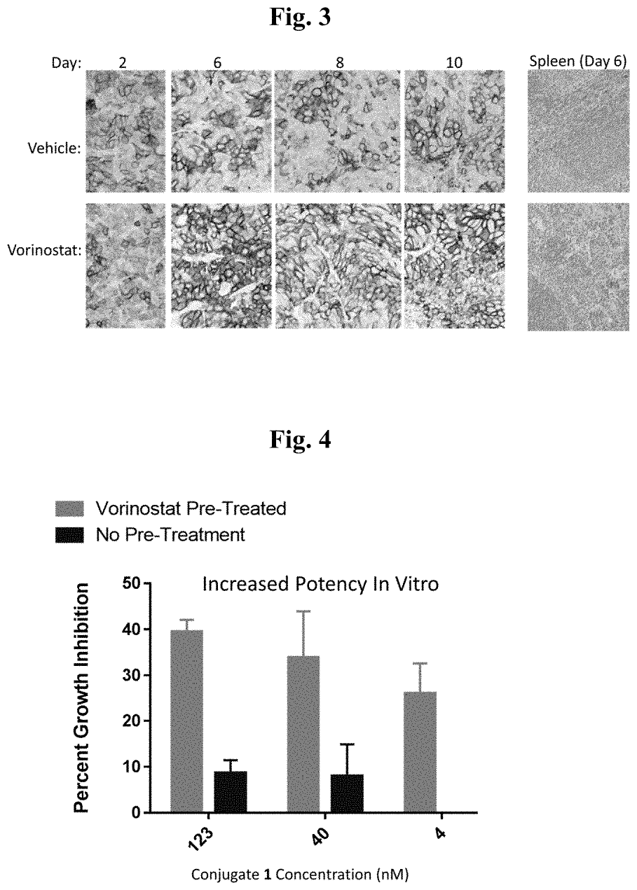

As shown in FIG. 3, increased SSTR2 expression was observed as a result of Vorinostat treatment. On Day 6 the IRS score doubled from 6 in the vehicle to 12 in the treated group. The immunoreactive score (IRS) is determined by multiplying the staining intensity in four gradations with the percentage of positive cells in five gradations.

Overall, tumors showed a more uniform expression pattern in comparison to the heterogenous expression pattern displayed in vehicle control group. However, an increase in expression was not observed in normal spleen tissues.

In a further study, whether increased SSTR2 expression would result in better delivery of Conjugate 1 to small cell lung cancer cells (NCI-H69) was evaluated. Conjugate 1 potency with Vorinostat pre-treatment was compared with Conjugate 1 potency without Vorinostat pre-treatment. As shown in FIG. 4, an increase in Conjugate 1 potency was observed with Vorinostat pre-treatment in vitro. Vorinostat alone had no effect on NCI-H69 cell proliferation (error bars represent STD values).

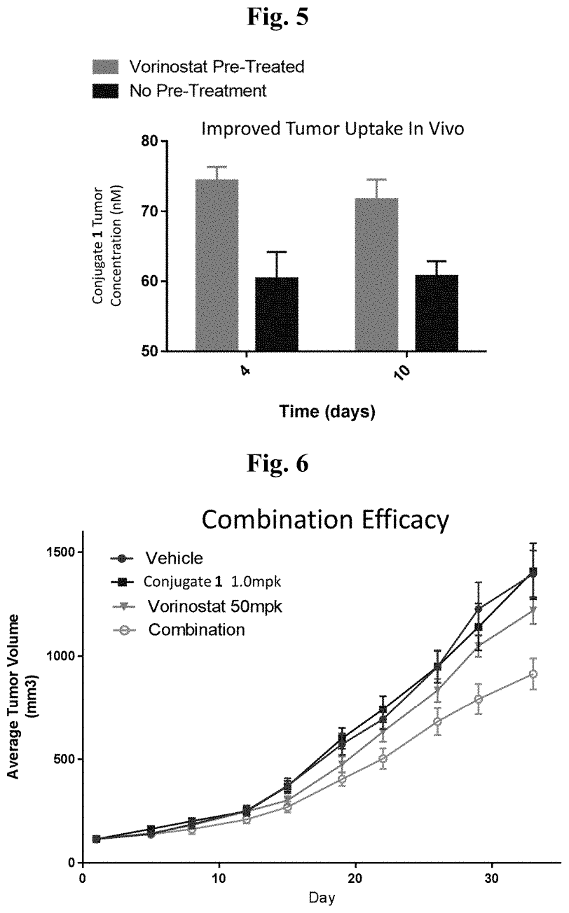

In vivo, mice bearing NCI-H69 tumors were pre-treated with Vorinostat then administered Conjugate 1. Tumors were analyzed for Conjugate 1 concentrations. As shown in FIG. 5, a statistically significant (P=0.03) increase in Conjugate 1 concentrations was observed as a result of Vorinostat pre-treatment (error bars represent SEM values).

### Example 7. Antitumor Efficacy of Conjugate 1 in Combination with Vorinostat

In a study with NCI-H727 model, Conjugate 1 and Vorinostat combination was tested in a tumor model that is not sensitive to single agents alone.

NCI-H727 (lung carcinoid) cell expresses very low levels of SSTR2 with an overall immunoreactive score (IRS) score of 1. Due to the limited SSTR2 expression in this model, Conjugate 1 shows no activity as a single agent. However, when Conjugate 1 is combined with Vorinostat, with treatment beginning on the same day (Conjugate 1 1× week via intravenous injection (IV) for 5 weeks (a total of 5 doses) and Vorinostat 5 days a week via intraperitoneal injection (IP) for 5 weeks (a total of 25 doses)), tumor growth inhibition was achieved as shown in FIG. 6 (error bars represent SEM values).

In a further study with NCI-H69 model (small cell lung cancer), low doses of Conjugate 1 were given to tumor bearing mice and tumor regression was tracked. 1/3 MTD of Conjugate 1 is 0.7 mpk. 1/6 MTD of Conjugate 1 is 0.35 mpk.

Mice bearing NCI-H69 tumors were treated with a combination of Vorinostat and Conjugate 1 starting on the same day or pre-treated with Vorinostat for 4 days IP then administered low doses of Conjugate 1. In total, mice received 10 total doses of Vorinostat (q.d×5) and 2 doses of Conjugate 1 (q.w.×2).

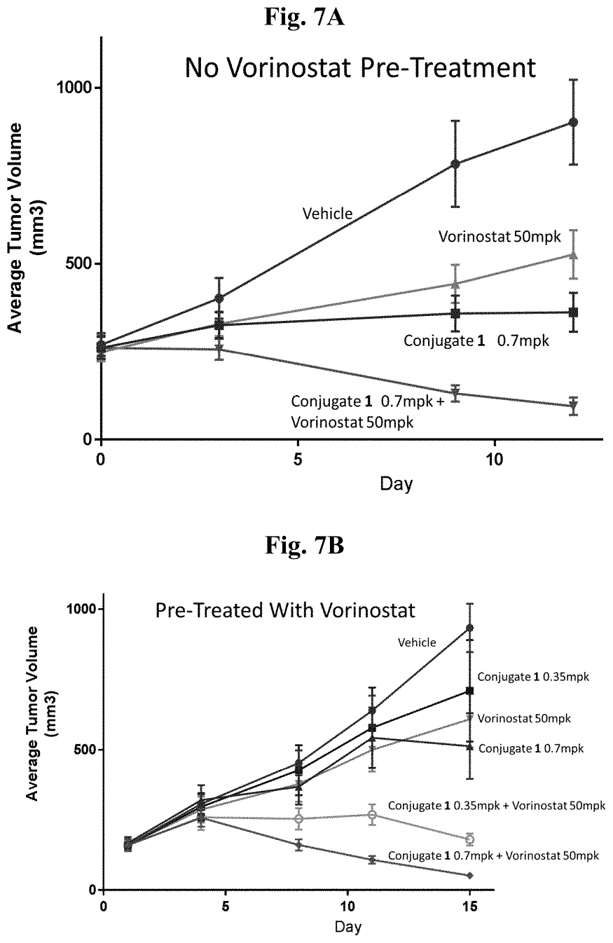

As shown in FIG. 7A and FIG. 7B, pre-treatment with Vorinostat increased the number of regressions observed in the 0.7 mpk (⅓ MTD) Conjugate 1+ Vorinostat group from 5 mice out of 10, to 10 mice out of 10. Additionally, tumor growth inhibition greater than 80% was observed at 0.35 mpk (⅙ MTD) of Conjugate 1 (error bars represent SEM values). No significant body weight loss was observed during these studies. Therefore, pre-treatment with Vorinostat resulted in better efficacy than the combination treatment alone due to increased SSTR2 expression as a result of Vorinostat pre-treatment, even at a lower dose of Conjugate 1.

All these data demonstrate that combination of Conjugate 1 and epigenetic modulators, such as HDAC inhibitors, provide greater efficacy than single agent activity alone and support the further evaluation of such combinations.

The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

1. A method of treating cancer of a subject, comprising administering: (A) a first component which comprises Component I, or a prodrug, derivative, or pharmaceutically-acceptable salt thereof; and (B) a second component which comprises Component II, or a prodrug, derivative, or a pharmaceutically-acceptable salt thereof, wherein
Component I is a conjugate comprising an active agent or prodrug thereof attached to a targeting moiety, wherein the active agent comprises a tubulin inhibitor or prodrug thereof; and
Component II is different from Component I.

2. The method of claim 1, wherein the targeting moiety of Component I binds to a somatostatin receptor (SSTR).

3. The method of claim 2, wherein the targeting moiety is selected from the group consisting of somatostatin, octreotide, seglitide, vapreotide, Tyr³-octreotate (TATE), cyclo(AA-Tyr-DTrp-Lys-Thr-Phe) where AA is α-N-Me lysine or N-Me glutamic acid, pasireotide, lanreotide, or analogs or derivatives thereof.

4. (canceled)

5. (canceled)

6. The method of claim 1, wherein Component I comprises TATE as the targeting moiety and DM1 as the active agent.

7. The method of claim 1, wherein Component I is Conjugate 1 having a structure of

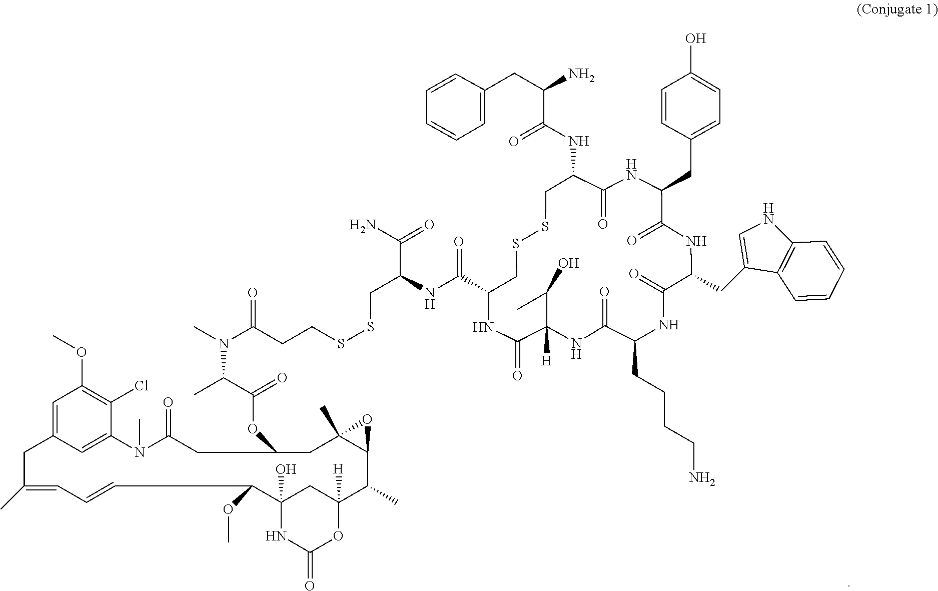 [SMILES: COc1cc2cc(c1Cl)N(C)C(=O)C[C@H](OC(=O)[C@H](C)N(C)C(=O)CCSSC[C@H](NC(=O)[C@@H]1CSSC[C@H](NC(=O)[C@H](N)Cc3ccccc3)C(=O)N[C@@H](Cc3ccc(O)cc3)C(=O)N[C@H](Cc3c[nH]c4ccccc34)C(=O)N[C@@H](CCCCN)C(=O)N[C@@H]([C@@H](C)O)C(=O)N1)C(N)=O)C[C@]1(C)O[C@H]1[C@H](C)[C@@H]1C[C@@](O)(NC(=O)O1)[C@H](OC)C/C=C/C=C(\C)C2]

8. The method of claim 7, wherein Conjugate 1 is administered in a pharmaceutical composition with a pH between about 4.0 to about 5.0.

9. The method of claim 8, wherein the pharmaceutical composition comprises acetate buffer.

10. The method of claim 8, wherein Conjugate 1 has a concentration of between about 2.0-about 5.0 mg/mL.

11. The method of claim 8, wherein the pharmaceutical composition further comprises mannitol.

12. (canceled)

13. The method of claim 8, wherein the pharmaceutical composition further comprises polyoxyl 15 hydroxystearate.

14. (canceled)

15. (canceled)

16. The method of claim 1, wherein Component II is a checkpoint inhibitor.

17. The method of claim 16, wherein Component II comprises a CTLA-4 antagonist.

18. The method of claim 16, wherein Component II blocks the PD-1 and PD-L1/2 checkpoint pathway.

19. The method of claim 18, wherein Component II comprises a PD-1 antagonist or a PD-L1 antagonist.

20. (canceled)

21. The method of claim 1, wherein Component II is an HDAC inhibitor.

22. The method of claim 21, wherein the HDAC inhibitor is vorinostat.

23. The method of claim 1, wherein Component II is octreotide or its derivative/analog.

24. The method of claim 1, wherein Component II comprises a radioactive agent.

25. The method of claim 1, wherein the subject further receives radiation therapy.

26. The method of claim 1, wherein Component II is a conjugate comprising an active agent or prodrug thereof attached to a targeting moiety, wherein the targeting moiety binds to heat shock protein 90 (HSP90).

27. The method of claim 26, wherein the targeting moiety of Component II is selected from the group consisting of ganetespib, geldanamycin, IPI-493, macbecins, tripterins, tanespimycins, 17-AAG, or a tautomer, analog, or derivative thereof.

28. (canceled)

29. The method of claim 26, wherein the active agent of Component II is selected from the group consisting of SN-38, irinotecan, lenalidomide, vorinostat, 5-Fluorouracil (5-FU), abiraterone, bendamustine, crizotinib, doxorubicin, pemetrexed, fulvestrant, topotecan, Vascular Disrupting Agent (VDA), or a fragment, derivative, or analog thereof.

30. The method of claim 26, wherein Component II is Conjugate 2 having a structure of 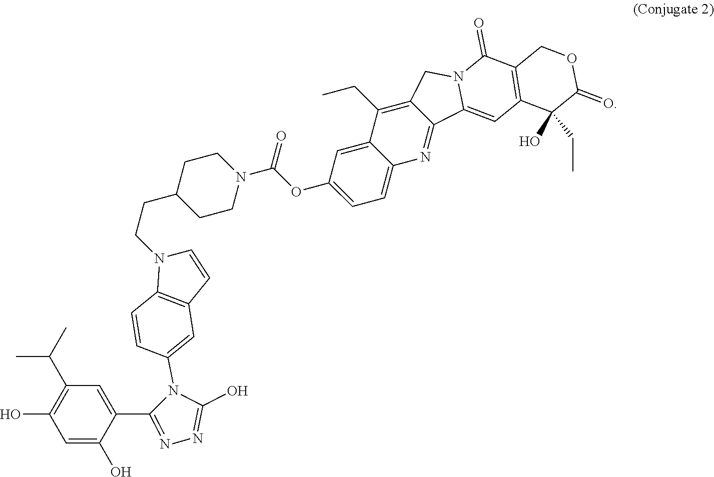 [SMILES: CCc1c2c(nc3ccc(OC(=O)N4CCC(CCn5ccc6cc(-n7c(O)nnc7-c7cc(C(C)C)c(O)cc7O)ccc65)CC4)cc13)-c1cc3c(c(=O)n1C2)COC(=O)[C@]3(O)CC]
or its tautomer.

31. The method of claim 30, wherein Conjugate 2 is administered in a pharmaceutical composition with a pH of above 8.0.

32. The method of claim 31, wherein Conjugate 2 is in its carboxylic acid salt form.

33. The method of claim 31, wherein the pharmaceutical composition further comprises sodium chloride.

34. (canceled)

35. The method of claim 1, wherein Component I is Conjugate 1 and Component II is Conjugate 2.

36. The method of claim 1, wherein Component I is Conjugate 1 and Component II is vorinostat.

37. The method of claim 36, wherein vorinostat is administered before Conjugate 1.

38. (canceled)

39. (canceled)

40. (canceled)

41. The method of claim 1, wherein the cancer is selected from a group consisting of lung cancer, breast cancer, colorectal cancer, ovarian cancer, pancreatic cancer, colorectal cancer, bladder cancer, prostate cancer, cervical cancer, renal cancer, leukemia, central nervous system cancers, myeloma, and melanoma.

42. The method of claim 1, wherein the cancer is a neuroendocrine cancer.

43. The method of claim 1, wherein the cancer is SSTR positive.

44. The method of claim 1, wherein the cancer is low in SSTR expression.
